(19) United States
(12) Patent Application Publication
(10) Pub. No.: US 2013/0198891 A1
(43) Pub. Date: Aug. 1, 2013

(54) METHOD FOR OBTAINING A WHEAT PLANT WITH IMPROVED YIELD PROPERTIES AND A NEW TYPE OF WHEAT OBTAINED BY SAID METHOD

(71) Applicant: Enrique JORGE THOMAS, Madrid (ES)

(72) Inventor: Enrique JORGE THOMAS, Madrid (ES)

(73) Assignee: MEGASEED S.A., Buenos Aires (AR)

(21) Appl. No.: 13/733,145

(22) Filed: Jan. 3, 2013

Related U.S. Application Data
(63) Continuation of application No. 10/585,977, filed on Jul. 13, 2006, now abandoned.

(30) Foreign Application Priority Data
May 10, 2005 (ES) PCT/ES05/00254
Jun. 8, 2006 (ES) PCT/ES05/00327

(51) Int. Cl. A01H 1/06

(52) U.S. Cl. CPC A01H 1/06
USPC 800/270; 800/320.3; 435/410; 800/302; 800/301

(57) ABSTRACT

The description of a method to obtain a wheat plant with improve yield properties that consists in the generation of genetic variability by means of the following stages: a) the construction of a wheat plant FI by means of the crossing of two genetically distant parents and of opposed industrial qualities; b) the permanent application through the whole development of the inflorescence of said plant of a high concentration of sunlight without spectrum filtration; and c) the germination of the resulting seeds and the analysis of the descendants for the search of stable variants having different chromosomal number. Also, the description of wheat plants obtained in this way that preferably have crown root, high production capacity of fertile shoots, shoot capacity, perennial habit, high level of grain productivity, high protein level and industrial qualities similar to the hard wheat of best quality, that qualify to be designated as a new type of wheat commonly called Megawheat. The productivity level of the wheat plants described exceeds a 60% the production of the conventional wheat and yield seeds with a weight over 55 g, preferably over 70 g.

### CROSS REFERENCE TO RELATED APPLICATIONS

This is a Continuation of Ser. No. 10/585,977, filed 13 Jul. 2006, which claims priority to PCT International Application Serial No. PCT/ES05/00327, filed 8 Jun. 2005, which claims priority to PCT International Application Serial No. PCT/ES05/000254 filed 10 May 2005. Each of the above references is incorporated herein by reference in its entirety.

### FIELD OF THE INVENTION

This invention relates to a method for obtaining a wheat plant with improved yield properties, a high grain productivity level and high protein level, and with similar industrial characteristics as the high quality hard wheat grains obtained by mutagenesis. The present invention is also related to the new wheat plant commonly called Megawheat, said plant has improve qualities such as a high grain productivity level.

Specifically, this invention is related to the field of wheat improvement, especially of Triticum aestivurn L., and to create a genetic variability to obtain a new type of wheat having special characteristics.

### BACKGROUND OF THE INVENTION

The wheat plant is one of the world's most important cultivated plant and its improvement have been practiced in empirical form since past times and in scientific form since the beginnings of the 20^{th} century in order to obtain better progeny. As a result, its adaptability to the most diverse sow latitudes and ecological conditions is remarkable (Curtis, 2002).

The fundamental significance of wheat in the bread making industry makes that its 25 yield by surface unit, flour yield, proteinic rate and the profile of the different proteinic fractions of the grain, represent the three main characteristics to consider in order to making a genetic improvement.

These characteristics are different depending on their objectives: therefore, the requirements for a good wheat to make bread are different than the requirements of a good wheat to make cookies. In this way, the grounds of any wheat improvement program is based in the availability of a large amount of germplasm that can be use to meet those different objectives.

In the next ten years a 30% increase of the international wheat demand is estimated. Therefore, it is strategic to diversify and improve the quality of the wheat production in order to be competitive with the exporter countries and satisfy the international demand at lower prices.

The yield of wheat is the result of the number of grains per surface unit and of the weight reached by the grains. Many authors have emphasized the importance relative to the component number of grains per surface unit in the wheat production. However, as it is explain next, most of the studies were directed at improving the bread quality of the flour by modifying the protein levels and its properties; but the achievements reached in improving de grain productivity level have been very low.

The cultivated wheat belongs to two different species: Triticum aestivum L. and Triticum turgidum L. ssp. (Desf.) Husn. The first of said species comprises four commercial kinds (hard red winter, hard red spring, soft red winter and white) and, the second comprises the wheat used for noodles. The wide variability allows wheat be the raw material for a variety of food products: bread, cookies, crackers, etc.

Since 1920 it was known that all the cultivated species from the genus Triticum could have three chromosomes: 2n=14; 2n=28 and 2n=42. This suggested a basic number of chromosomes of 7 and the resulting appearance during the evolution of the genus of the diploid species (2n=2x=14), tetraploids (2n=4x=28) and hexaploids (2n=6x=42).

All the studies have confirmed that 1x=7 is the basic chromosomal number of the tribe Triticeae. Also, each specific chromosome or part of a chromosome of the base genome is specifically related with a chromosome or part of a chromosome in another genome of the tribe Triticeae.

The bread wheat (Triticum aestivum) is a allohexaploid with three genomes, B, A and D (genomic formula according to Waines and Barnhart, 1992). Each genome derives from a different specie: the B genome comes possibly from an Aegilops speltoides Tausch ancestor; the A genome derives from Triticum urartu Tum. Ex Gand; and the D genome comes from Aegilops tauschii Coss (Kihara, 1944; McFadden and Sears, 1946).

The Aegilops genus has contributed with two thirds of the modern Triticum genome (Wheat Genetics Resource Center, 2004) and is the source of the B and D genomes of bread wheat. Studies made by varying isoenzymes (Jaaska, 1978), nuclear DNA (Dvorak and Zhang, 1990) and organelle having DNA (Mori and col., 1988; Wang and col, 1997), strongly support the idea that the B genome comes from species with the S-genome of the Sinopsis section, closely related to the alogama species Aegilops speltiodes.

Each one of the three genomes (B, A and D) of the bread wheat is composed by 7 chromosomes, called 1 B, 1A, 1D, up to 7B, 7A and 7D, respectively. In that way it is possible to locate the 21 different chromosomes in three groups. The chromosomes of each group are called homologous(=similar) and it is considered that they have common evolution origins (Kimber and Feldman 1987). The term homologous is referred to the pair of chromosomes in a genome that have the alleles for the same genes.

Cultivated wheat is considered a species with less genetic variability than other cultivated plants such as barley or corn due to its relatively recent evolutionary origin (Sharp, 2004).

The methods used for wheat improvement, as well as the methods used for the rest of the cultivated plants, are varied go from classic techniques of conventional improvement describe in widely known studies (ej., Allan, 1987; Allard, 1960; Heyne, 1987; Simmonds, 1979), including marker-assisted improvement and genetic engineering (el. Hoisington, 2002).

However, none of the studies reported methods to improve the production of wheat grain per surface unit and with low production costs.

Since hundred of years ago there have been a search for mutations in order to find wheat variations that provide flour with the adequate quality for the bread industry, which is its final destiny. These mutations can be done by an artificially induced method, being this method one of the most efficient for the generation of genetic variability which, combined with the selection and recombination of the adequate lineage, could give way to genotype of commercial interest.

The mutations constitute hereditary alterations of the genetic material and can affect entire chromosomes in a way that the “new” individual has a different chromosomal number than the original individual, or they can be point mutations, invisible cytologically, which imply changes in the constitution of the nucleotides in the DNA.

The mutations can be induced in the whole plant or in in vitro cellular cultures. The procedures used up to now utilize the following types of mutagenic agents (Montelone, 1998):
a) Chemical: the different types of chemical mutagenic are known by their reaction mode, and they can be:
   a. Analog of Base agents: these compounds are chemically similar to the purine bases and the pyrimidine bases in the DNA. For example, Bromouracil, aminopurine.
   b. Agents capable of alter the structure and pairing of the DNA bases. For example, nitrous acid, nitrosoguanidine, methyl-methane-sulfonate, ethyl-methane-sulfonate.
   c. Intercalating agents. For example, Acridine orange, proflavine, ethidium bromide.
   d. Agents that alter the DNA structure. For example, NAAAF, psoralenes, peroxides.
b) Physical/Radiation: Radiation was the first known mutagenic procedure and the first report goes back to the year 1920. The radiation was discovered in 1890 and Roetgen discovered the X-rays in 1895. The radiations are classify in:
   a. Ionic: Radiation in which the displace particles are ions. These types of radiation are the more harmful to one's health: X-rays, gamma rays, alpha particles, beta particles and neutrons, i.e., nuclear energy. This type of radiation has an energy capable to produce reactive ions (atoms or charged molecules) when they react with biological molecules (for example DNA), therefore it is called ionic radiation.
   b. Non-ionic: Radiation in which there is no intervention form ions. An ion is defined as an atom that has lost one or more electrons. For example: ultraviolet radiation, visible radiation, infrared radiation, lasers, microwaves, and radiofrequency. Ultrasound can be included here since the risks produced are similar to the ones produced by the non-ionic radiation. Non-ionic radiation includes infrared, the visible spectrum, and ultraviolet. These types of radiation have less energy than ionic radiation, even though they are capable of altering biological molecules like the DNA.

Ionic rays are mutagenic agents that induce especially important restructures in the genetic material. These radiations have been used in vegetables like corn (Fujii, 1978), in the legume Medicago truncatula (Sagan et al., 1995), and in the model plant Aradidopsis thaliana (Redei, 1974).

The types of mutations observed are deletion, translocations, inversions, insertions and point mutations. These types of mutations are not necessarily exclusive, for example, a deletion can also have an insertion or an inversion (Shirley et el., 1992). However, the dominant type of mutation produced with this type of radiation are the deletions (>75%).

The search for “major” hereditary alterations such as the loss of chromosomal parts from a chromosome or from a complete set of chromosomes cannot be considered a logic goal to search in all the cultivated plant species. However, wheat shows a special feature, since due to polyploidy, its genome is consider of the buffer type in which it is possible to introduce chromosomal parts, whole chromosomes or large linkage groups (for example, the triticale case), and also the deletion of chromosomal parts or of whole chromosomes. Since a long time ago it has been perfectly demonstrated that the wheat genome can stand a high karyotype instability that does not necessarily conclude in a speciation phenomena, even though it could be possible.

Ernest Robert Sears developed, at the beginnings of the fifties, a method to obtain monosomics in wheat (i.e., 2n−1=41 chromosomes) and from them obtain nulisomics (2n−2=40 chromosomes). However, the obtaining rate of nulisomics was very low (0.3). Even with this trouble, Sears was capable of obtaining nulisomics for each one of the 7 basic chromosomes of bread wheat and describing its general morphologic characteristics. In the last one hundred years there have been done and studied wide direct crossings and the later selection between related species or progenitors of bread wheat in the Triticeae tribe (see revision of Mujeeb-Kazi and Rajaram, 2002). The first of these hybrids was between wheat and rye obtained by Wilson in 1876. Later, and working in the same type of crossing, Rimpau described 12 plants of Triticale in 1891.

However, in the particular case of the modifications of the chromosomal number of wheat, the nulisomics described by Sears and the cases reported by Mujee-Kazi and Rajaram, even though they are a valuable tool for cytogenetic studies, they have not have any commercial application basically due to their agronomic deficiencies and poor yield.

Later, Farrer obtained hybrids in 1904 between wheat and barley. Since 1930 and on, many interspecific and intergeneric crossing experiments in the Triticeae tribe were performed with the purpose of transfer perenniality to the wheat, basically between Triticum and Aegilops. There are about 325 species of the Triticae tribe, of which approximately 250 are perennials and 75 are annuals, and in the last one are the bread wheat, hard wheat, triticale, barley and rye (Dewey, 1984). Very few wide crossing experiments between perennial and annual species have been successful due to the high complexity to do this and to the failure of the embryo culture technique. The perennial species more used in these experiments belong to the Thinopyrum group and, in general, there were used fodder species with valuable characteristics from their sickness resistance point of view.

Therefore, there is still the need to provide a method for obtaining wheat plants having a high grain production but with an agronomic quality similar to the best wheat commercially available. There is also the need to obtain new wheat plants by a procedure with substantially reduced processing times and energy costs.

### SUMMARY OF THE INVENTION

The first object of the present invention is to provide a method for obtaining a modified wheat plant with improved yield properties, a high level of grain productivity, a high protein level and similar industrial characteristics than the high quality hard wheat.

More specifically, it is a main object of the present invention to provide a method for obtaining genetic variability in wheat, especially in Triticum aestivum L., with improved production qualities, high level of grain productivity, high protein level but with industrial characteristics similar to high quality hard wheat.

Another object of the present invention is to obtain a modified wheat plant with improved yield properties, high level of grain productivity, high protein level and industrial characteristics similar to high quality hard wheat by a simple and economic method, with reduced processing times and energy costs.

Another object of the invention is to obtain a modified wheat plant that provides wheat grains with a weight of 1000 grains over 55 g, preferably over 70 g.

More preferably, another object of the invention is to obtain a wheat plant having a crown root, a high fertile shoot production capacity, a shoot capacity giving a perennial habit and a high grain productivity.

It is another object of the present invention to provide a method to obtain a greater wheat grain production that is economic with processing times and energy costs substantially reduced by obtaining a genetic variability in a wheat plant.

Another object of the present invention is a method for generating genetic variability in a wheat plant that provides wheat with improved characteristics.

The present invention also provides a modified wheat plant or parts of a modified wheat plant having a crown root, a high fertile shoot production capacity, a shoot capacity and perennial status that gives a high level of grain productivity, high protein level and industrial characteristics similar to the best quality hard wheat.

The authors of the present invention have discovered that is possible to improve the wheat grain production obtained by surface unit generating a genetic variability in the wheat, preferably in Triticum aestivum, by a simple method that comprises the permanent application along the whole development of the inflorescence of a high concentration of sun light with no spectrum filter to a construction of a plant F1 obtained by crossing of two genetically distant parents and of opposed industrial characteristics; followed by the germination of the resulting seeds and the analysis of the descendants for the searching of stable variants.

By means of this process, the authors of the present invention have achieved to generate genetic variability in wheat, having the common name of Megawheat. The genetic variability is showed in the presence of crown root, high fertile shoot production capacity, shoot capacity, perennial habit, high level of grain productivity, high protein level, and industrial characteristics similar to the best quality hard wheat. More specifically, the advantageous and amazing characteristics of the new wheat plant Megawheat obtained by the process of the present invention are the presence of crown root, high fertile shoot production capacity, shoot capacity, and perennial habit that without holding any particular theory they provide a high level of grain productivity and high protein level.

The wheat plant obtained by the method of the present invention provides a remarkably improved yield with respect to other varieties available in the market. Therefore, one of the characteristics of the wheat plant obtained by the process of the present invention that contributes to higher yield is that the wheat grains remarkably exceeds in weight the commercially available wheat grains. The average weight of each wheat grain obtained by the process of the present invention is approximately between 50 and 80 grams, more preferably 70 grams; while the maximum weight of the commercially available wheat grains is about 45-50 grams. Furthermore, the wheat plant obtained by the process of the present invention has an average of 20 spikelets per spike compared with 14 spikelets per spike in the commercially available wheat. Each one of these spikelets yields approximately between 3 and 4 wheat grains per spike. Therefore, the greater weight of the wheat grains obtained by the process of the present invention in addition to the larger amount of grains produced by plant provides a remarkably improved yield in the wheat production. As the Comparative Test of Yield will demonstrate in detail by the use of the software Infostat (Infostat, 2004) it is possible to obtain a wheat grain production that exceeds in a 60% the production of the conventional wheat.

Another advantage presented by the new wheat plant obtained by the present invention is the shoot capacity from a reserve zone. The adult plants obtained this way, with a number of shoots form a base region having many stems proximate to each other and a large amount of adventitious roots produced in knots over the soil level. This proximity situation generates a kind of stem welding in the base part gives a situation of a reserve zone. As a result, it has been determined the development of about 150 shoots per plant, while in the usual densities there have been counted 15-20 shoots. Due to this formation, the new wheat plants obtained by the method of the present invention show a perennial life habit, meeting with all the known vegetative and reproductive phases. This property allows that, once the plant is grown, new plants can be obtained again and consequently a higher grain production can be obtained without the need of sowing seeds and without the need to employ more labor.

Therefore, the time and energy costs to obtain a better wheat grain yield are substantially reduced with the process of the present invention.

Moreover, the procedure of the present invention provides wheat grains having values in the quality standards of the bread making industry that establish them in the superior quality scale for the quality standards of Australia, Canada and the United States. The wheat grains obtained by the process of the present invention have a gluten pattern belonging to varieties with a very good bread-making quality; a gluten force comparable to the commercial wheat varieties of good bread-making capacity and also a protein content of approximately 40% superior to the commercially available wheat grains.

### DETAILED DESCRIPTION OF THE INVENTION

The present invention relates to a new method of improving vegetable applied to bread wheat, with a capacity to generate a genetic variability in said species. From the analysis of the descendants, an amount of plants was identified having innovative phenotype characteristics, characterize by the presence of crown root, high production capacity of fertile shoots, long and wide leaves and in some cases finely serrated, leaves having a central vein, shoot capacity, perennial habit, high level of grain productivity, high protein level and preserving industrial qualities similar to the high quality hard wheat.

The present invention also includes the wheat plant obtained by the process of the invention. As used here, the term plant includes but is not limited to the whole plant, plant cells, plant protoplasts, plant cell tissue cultures, plant callous, plant population, and parts of plants that are intact in the plant or part of plants such as embryos, pollen, ovules, flowers, grain, leaves, stem, root, anthers.

The present invention also includes the seeds produced by the wheat plants. Advantageously, the wheat line Megawheat can be used and crossed with other different line to obtain plants with superior characteristics. All the plants produced using the wheat line Megawheat as the progenitor are also an objective of the present invention.

The term wheat plant, as used in the present invention, refers to a plant that is a member of the Triticum genus. The wheat plants of the present invention may be members of the genus Triticum that includes but is not limited to T. aestivum, T. turgidum, T. timopheevii, T. monococcum, T. zhukovskyi and T. urartu and hybrids of the same. Examples of the subspecies T. aestivum that are includes in the invention are: aestivum, compactum, macha, vavilovi, spelta and sphaecrococcum. Examples of the subspecies T. turgidum includes in the present invention are: turgidum, carthlicum, dicoccon, durum, paleocolchicum, polonicum, turanicum and dicoccoides. Examples of the subspecies T. monococcum includes in the present invention are: aremonococcum and aegilopoides preferably in Triticum aestivum L.

The method to improve the wheat grain production obtained by surface unit in the present invention comprises the steps of:
a. The construction of a wheat plant F1 by crossing two genetically distant parents having opposed industrial qualities;
b. The permanent application of a high concentration of sun light without spectrum filtration during the development of the inflorescence of said plant;
c. The germination of the resulting seeds and the analysis of the descendants in order to find stable variants.

There is no known information about mutation inductions in superior vegetables by means of direct sunlight, i.e., visible plus non-visible spectrum (ultraviolet and infrared).

Solar irradiation comprises a part of the electromagnetic spectrum between 300 and 1500 nm. Here are included the visible spectrum and the luminous non-visible spectrum. The visible spectrum, also called the optical window, comprises approximately from 380 nm (purple), to 770 nm (red). Above 770 nm are the infrared radiations and below 380 nm are the ultraviolet radiations.

Infrared radiation was discovered by the astronomer William Herschel (1738-1822) in 1800, when he measured the high temperature beyond the red zone of the visible spectrum. The infrared band is divided in three sections: near (770-2500 nm), intermediate (2500-50000 nm) and far (50000-1 mm). Every molecule having a temperature over the absolute zero (−273° K) emits infrared rays which will be larger with the higher the temperature of the object.

The generation sources of the infrared radiation are: sunlight, incandescent bodies and very hot surfaces, flames, incandescent lamps, fluorescents, etc.

The biological effects of the infrared radiation are slight since, due to its low energy level, it does not react with live matter and it only produces thermical effects. The damages produced by infrared radiation may appear in the skin and in the eyes. The exposure to radiation may cause burns and increase skin pigmentation. The eyes have a defense mechanism, but the radiation might cause erythema, cornea injuries and burns.

The visible spectrum for the human eye of the solar irradiation was decomposed by Isaac Newton using a prism. The white light constitutes a combination of waves having similar energies and none of the waves predominate over the others. Visible radiation goes from 380 nm to 770 nm. The lower frequencies of the visible light (long wave longitude) are perceived as red and the higher frequencies (short longitude) appear purple.

For a long time ago it was believed that the visible spectrum of light had no mutagenic effect. However, it has been demonstrated that even this band of the spectrum has mutagenic activity in microorganisms (Kubitschek, 1967; McGinty and Fowler, 1982; Kielbassa et al., 1997; Voskanyan, 1990; Xiang Yang, 1990; Sinha et al., 2002).

Ultraviolet radiation (UV) is located between the X-rays and the visible light spectrum. UV radiation was discovered by Johann Wilhelm Ritter in 1801 when he achieve to obscure silver salts exposing them beyond the purple end of the visible light. UV radiation constitutes an important part of the light send by the Sun to the Earth. These rays have an energy that produce atomic ionization and as a result the ionosphere is formed on the Earth. This strong chemical effect makes them toxic for life getting them to produce carcinogenic mutations on the skin. Ozone is the substance in our atmosphere that absorbs part of the ultraviolet rays and prevents them getting to us.

The generation sources of UV radiation are: sunlight, germicide lamps, phototherapy lamps, UV-A solar lamps, weld and cut arcs, photocopiers, etc.

The biological effects of UV radiation are well documented and the same can react with DNA bases and aromatic amino acids from proteins being, therefore, an important mutagenic agent and even lethal in microorganisms (Montelone, 1998; Voskanyan, 1999). The UV radiation is normally classified based on its wave longitude and may be:
a) UV-C (180-290 nm). This is the most energetic and lethal and cannot be found in sunlight since it is absorbed by the ozone layer. However, the ozone layer depletion in some regions of the planet can let this type of radiation to reach the earth surface;
b) UV-B (290-320 nm). This is the fraction of sunlight with a higher lethal and/or mutagenic capacity;
c) UV-A (320 nm-visible). This is the so called near UV and can have some deleterious effects, primarily because it provokes the appearance of oxygen radicals and, second, because it produces pyrimidine dimers.

It is known that ultraviolet radiation may increase in some special circumstances. Foe example, it is known that when there is the development of clouds of the cumulus type, the same can act as mirrors and diffusers and increase the UV intensities and therefore, increase the solar risk. A not-so-thick layer of clouds can blocked the visible radiation (the shadows over the surface turn blurred) and the infrared radiation (the heat sensation is diminished) but not the ultraviolet radiation that will still getting to the surface and therefore it can cause burns. Some thin clouds can have the effect of a magnifying glass and some white clouds can act as a mirror reflecting the radiation and increasing the ultraviolet radiation that reaches the surface. At the same latitude the radiation received by the earth surface will be determined by the total atmospheric ozone, the cloudiness, the atmospheric pollutants, the level of the soil where we are and the type of soil. The snow, the sand and the cement act as mirrors and reflect the ultraviolet radiation, increasing locally the amount that we received. The height is also a factor: the higher we are, the higher the amount of UV radiation received (American Optometric Association, 2004; Environmental Protection Agency, 2004).

However, the irradiation of a biological material, in this case a wheat plant, can be performed by direct impact or by reflections of different characteristics that are produced naturally and continuously or artificially by irradiations with different energy sources or modifying the path of the incident natural radiation. The experimenter can try to generate changes in this way and select the individuals that suppose, have been favored or that register favorable changes. In this way the proportion of favorable transformations is very low and fortuitous.

In the case of the present application the experimenter intentionally modified the low energy incident radiation and, probably, the quality of the same by non-conventional means (reflecting with mirrors). This modification of the incident radiation applied from before of the beginning of the reproductive phase up to its end may lead hereditary modifications.

The first step of the invention carried out in the year 1997, consisted in building a F1 having a genotype capable of generating in a natural form a wide genetic variability in its descendants.

To generate this maximum variability prior to the mutagenic treatment there were selected two wheat varieties as progenitors to obtain the F1 (S0) belonging to the Thomas Farm in the Republic of Argentina having industrial qualities and divergent cycles:

a) The new line of Triticum aestivum L. of 2n=42 of internal denomination “Thomas 796” was used as the mother, and its origin can be trace from a segregate population of origin CIMMYT by its abbreviation in Spanish (International Center of Corn and Wheat Improvement), selected in Obregon-Sonora-Mexico in F2, of early cycle, tall appearance and quality of the “soft” type;

b) as the father it was used the Triticum aestivum L. variety of 2n=42, denominated “Thomas Aconcagua” of excellent industrial quality with high stability, of long cycle and short appearance, and its origin can be tracked in the following sketch:

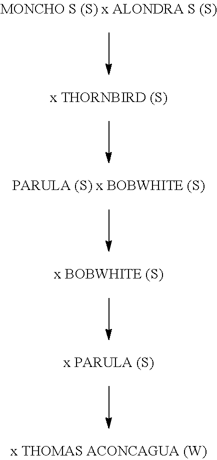

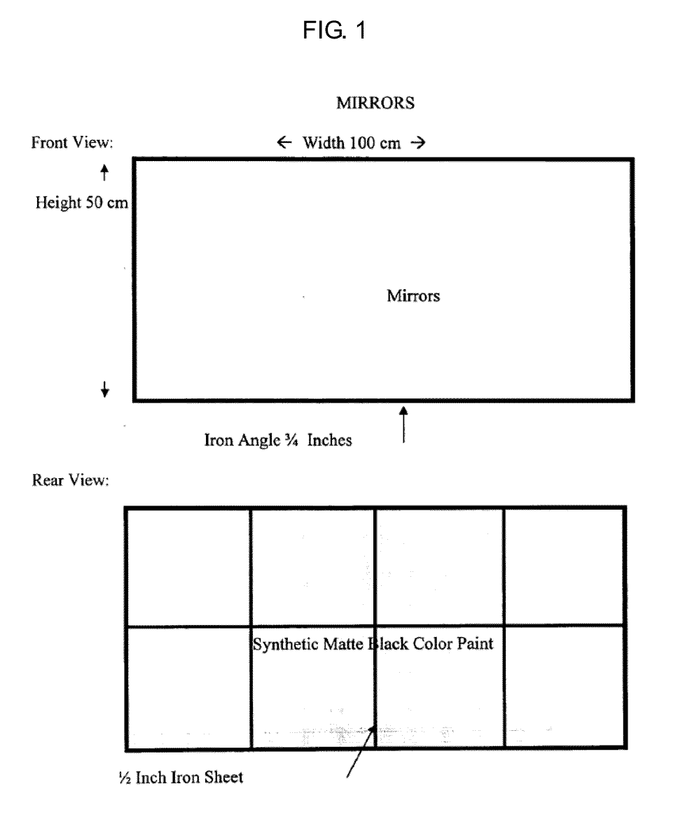

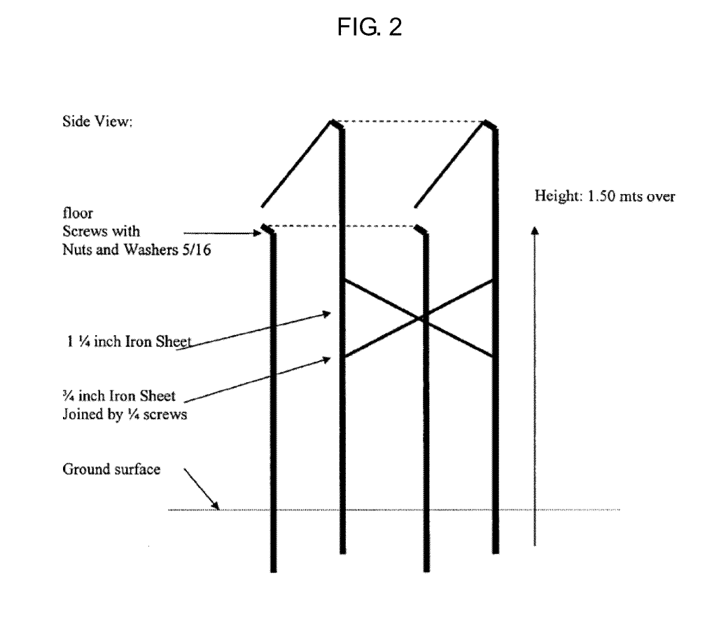

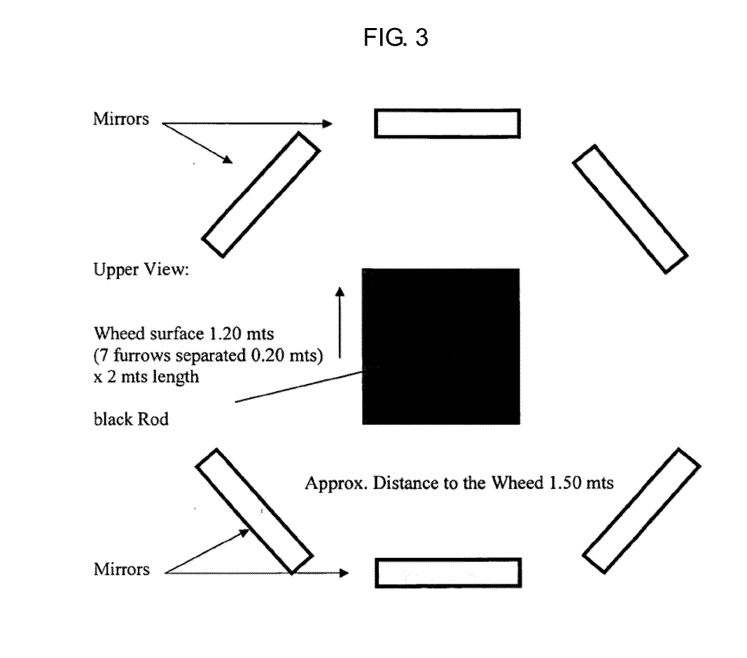

In 1998, F1 (S0) was sowed and from the moment of the glean to the blooming there were 100 plants submitted to a mutagenic process of a high concentration of solar rays with no wavelength filter and with the object of getting deep alterations in the DNA of the germinal cells:
a) The 100 wheat plants where sowed in one plot of 1.20 meters wide by 2 meters long in 7 furrows separated by 0.20 meters. The direction of the furrows was East-West;
b) 6 mirrored surfaces each of 1.00 meters long by 0.50 meters height were separated. Each one of these mirrored surfaces was formed from back to front by an iron square holding a mirror having its back part treated with synthetic paint of a matte black color (see FIG. 1);
c) The 6 mirrored surfaces were mounted on 6 supports in a way that the mirrors were pointing out by their center towards a stick placed equidistant in the middle of the plot with the plants (see FIG. 2);
d) Three of the mirrors were placed towards the west and three were placed towards the east. The distance from the mirrors to the wheat plot was 1.50 meters (see FIG. 3);
e) The direction of the mirrors varied according to the height of the plants.

In the year 1999, the 866 grown seeds F2 (S1 or M1), were sowed, and only two of then were germinated, producing two plants which produced 182 seeds that were grown as a group. It was noticed that the size of some of these seeds exceeded the normal size for the Triticum aestivum species since they weighted 12.8 grams and measured 9 mm long by 4mm wide.

In the year 2000, the 182 grown seeds F3 (S2 or M2) were sowed and it was noticed the appearance of plants having different forms, heights, cycles and sanitary behavior, and there was a selection based only in favorable phenotype aspect. Seventeen plants were selected and were grown individually.

In the year 2001, 17 individual plots were sowed with the grown seeds F4 (S3 or M3) from the 17 selected plants. The material showed again great plant segregation in terms of plant form, height, cycle and sanitary behavior, obtaining a new selection based in favorable phenotype aspects. Plants of each one of the 17 plots were used to do crossings with commercial varieties of bread wheat Triticum aestivum L. to make 17 crossing groups.

In the year 2002, there were sowed:
a) The seeds F1 of the 17 crossing groups, noticing that 16 of them germinated and only one group did not germinate because the crossing was unfeasible, and;
b) 825 total plots coming from the grown seeds F5 (S4 or M4) from the selected plants of the 17 plots of the previous year.

From this moment on, we will only refer to the plants of the plot which crossings F1 with Triticum aestivum L. did not germinate.

In the year 2003, 22 furrows were sowed with 100 seeds F5 (S5 or M5) coming from the 22 selected plants. All the plants continued to show a high segregation for cycle and plant height.

The remaining seeds of the 22 sowed furrows were taken to the Botanic Department of the Faculty of Agronomy of the Central University of the Buenos Aires Province Republic of Argentina in order to make a chromosomal count. The study allow to check that in no case the chromosomal number was normal for the species Triticum aestivum L. of 2n=42, having obtained chromosomal numbers of 2n=32 to 40. This explains why the crossings with Triticum aestivum L. of 2n=42 chromosomes were unfeasible.

When the chromosomal count is correlated with the phenotype characteristics of the 22 sowed furrows, it was clear that in the four furrows with plants of 2n=40 chromosomes, they shared the following characteristics:
a) Ears of a size much bigger than the normal for the species Triticum aestivum L;
b) Roots in crown;
c) High production capacity of fertile shoots;
d) Leaves longer and wider than the normal for the species Triticum aestivum L.;
e) In some cases, finely serrated leaves, and;
f) Leaves with a central vein.

The plants from the four selected furrows of chromosomal constitution 2n=40 were grown by individual ear.

A bulk was made having in a proportional manner a part of the grown progenies of all the seeds in order to have an enough amount to make different characterization analysis. This bulk is called MEGAWHEAT.

The MEGAWHEAT seed was used for the following analysis:

1) Morphologic Description of the Megawheat

The following morphologic description of the Megawheat plants was made in the Botanic Department of the Faculty of Agronomy of the Central University of the Buenos Aires Province of the Republic of Argentina in the year 2004, with Megawheat plants sowed on Jul. 13, 2004, with emergence on Aug. 3, 2004.

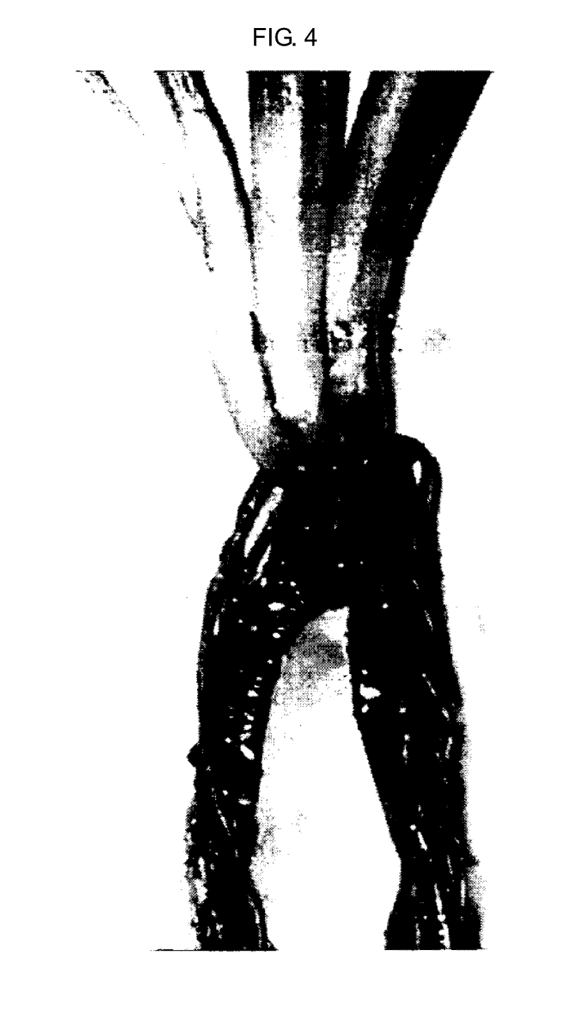

Herbaceous plants that with germination show coleoptiles of a white color and with no antocianic pigmentation (FIG. 4: picture of coleoptile).

The plants show foliar anatomy corresponding to a type C3 similar to the winter gramineae.

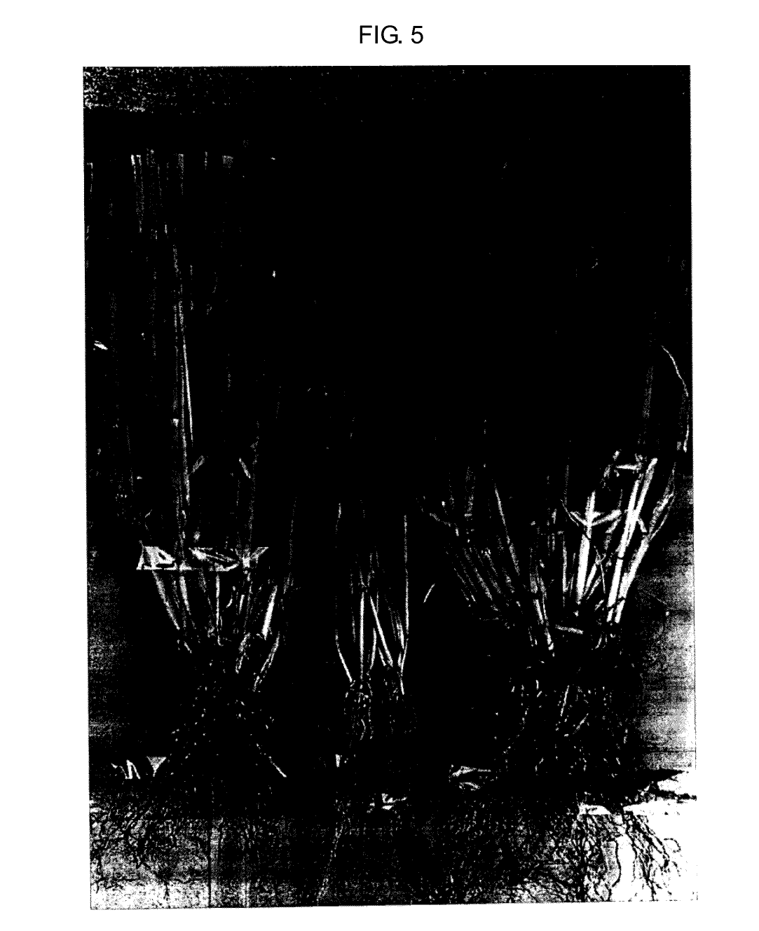

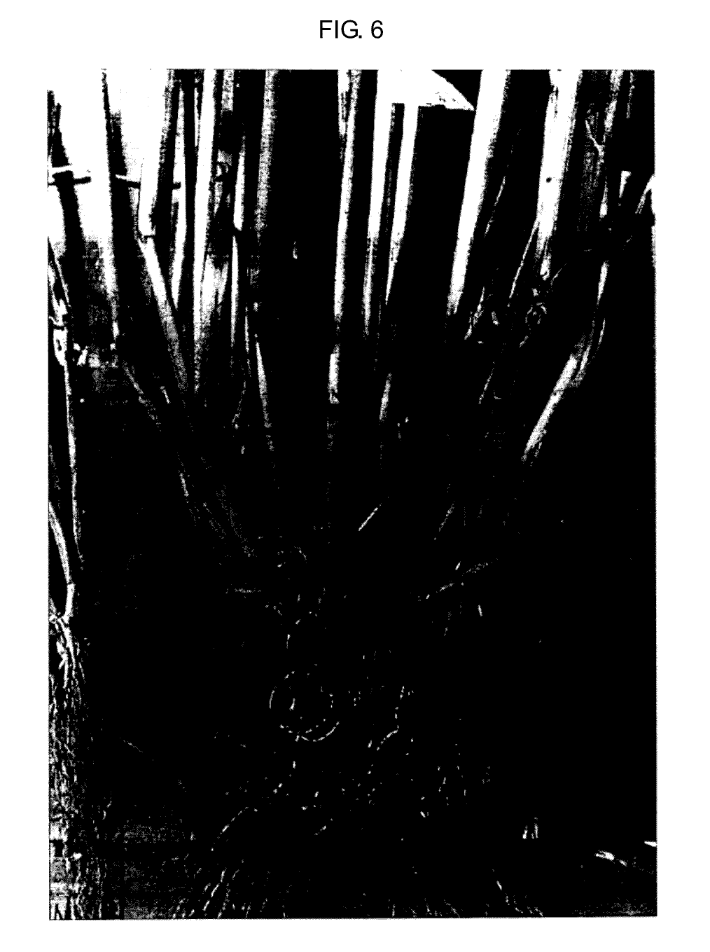

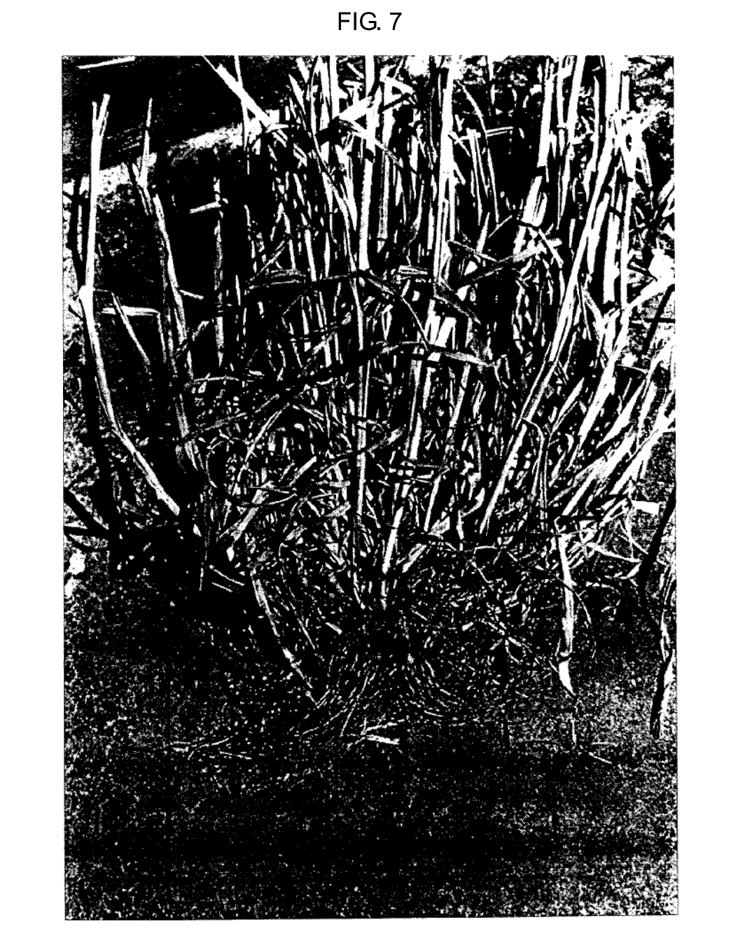

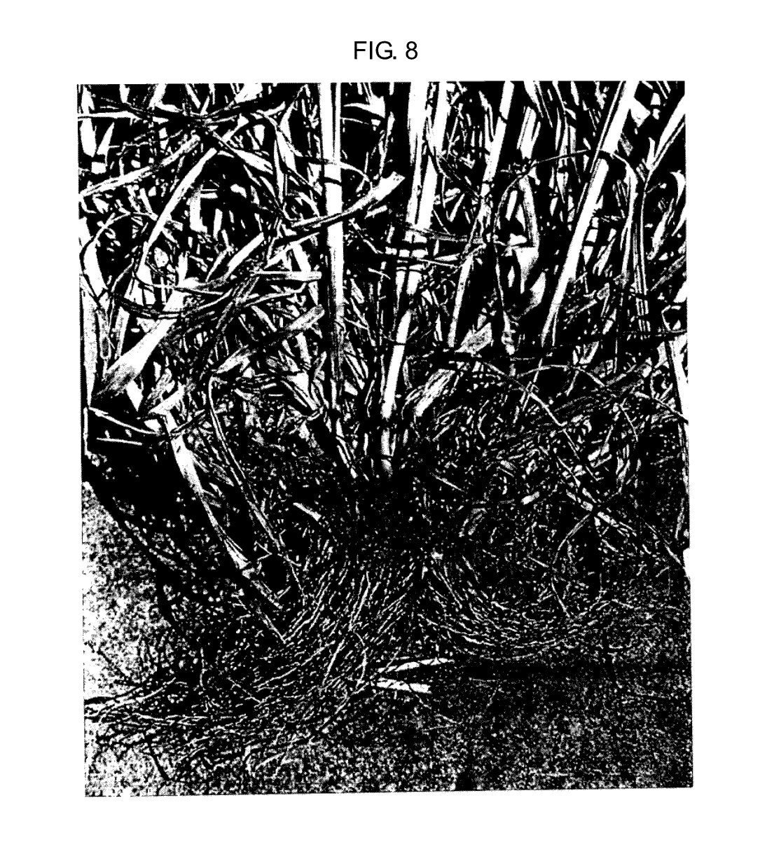

The adult plants vary from 0.78 to 1.60 meters of height with many shoots (FIG. 5: picture of shoots and whole plants), that cause a basal region constituted by many stems close to each other (FIG. 6: pictures of stems close to each other), and a great amount of adventitious roots produced in knots over the soil level (FIG. 7: pictures of roots). This proximity situation causes a kind of stem welding in the basal part and gives a reserve zone situation (FIG. 8: another picture of roots showing the reserve zone).

As a result, it has been determined, in specimens sowed with low density, the development of approximately 150 shoots per plant, while in the usual densities there were counted 15-20 shoots.

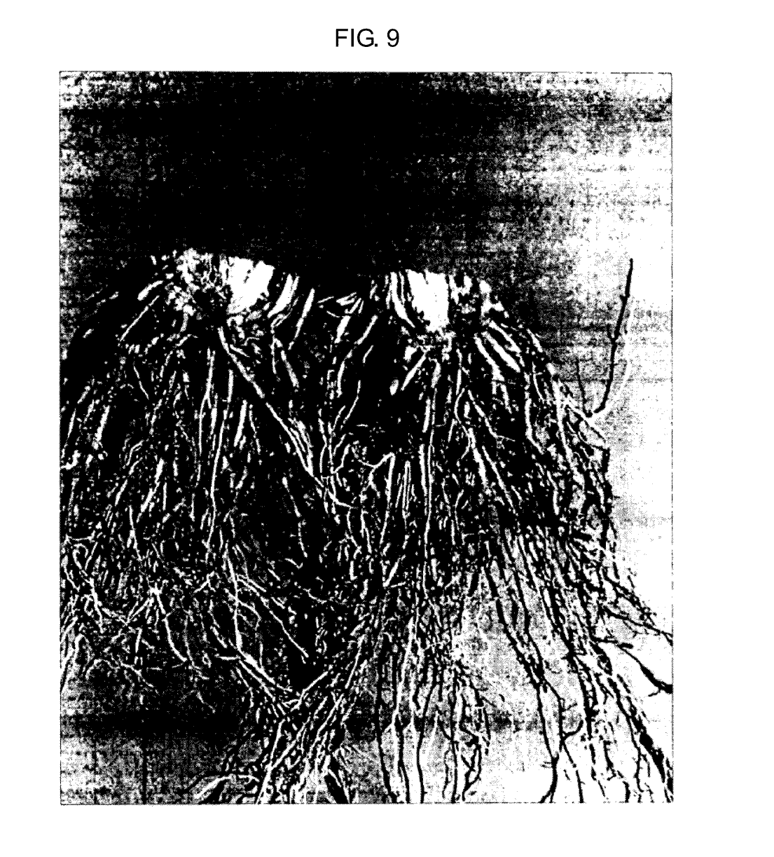

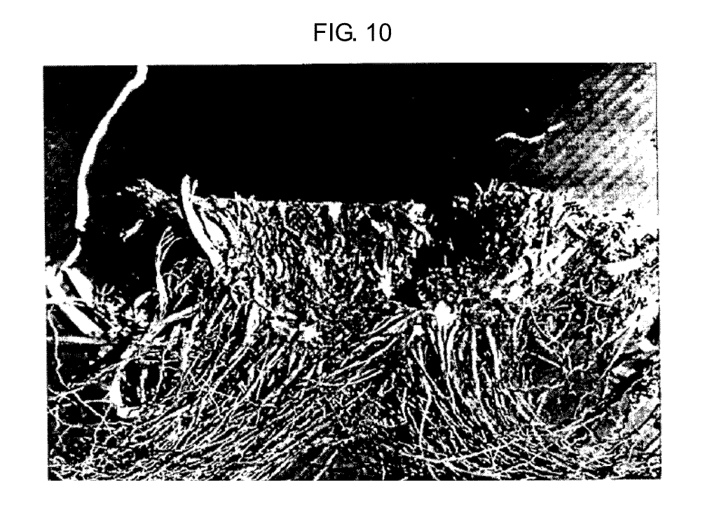

In the dissection of adult plants it was verified that in 40 days from the emergence, in the test conditions, a species of basal crown started to form (FIG. 9: another picture of roots showing the basal crown). FIG. 10 shows a subsequent state.

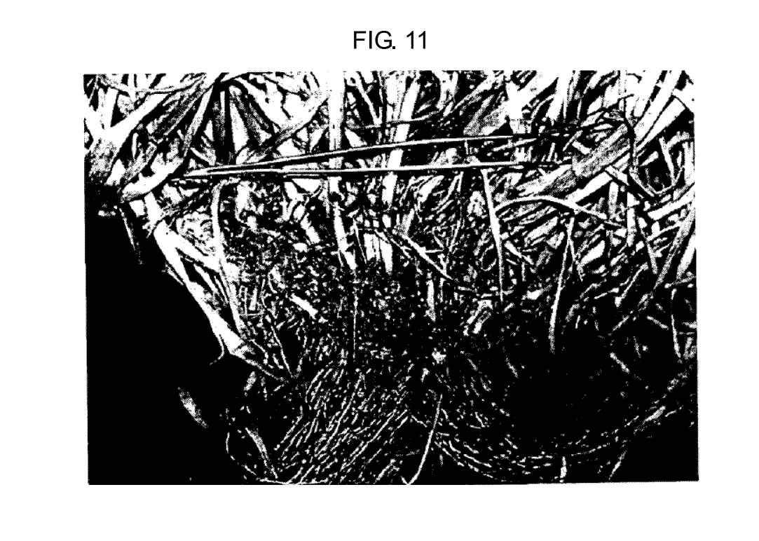

This characteristic is similar to the one describe for the genus Aegilops, considered an ancestor of Triticum aestivum (Morrison et al., 2002). FIG. 11 shows a detail of the undersurface system of the material in study.

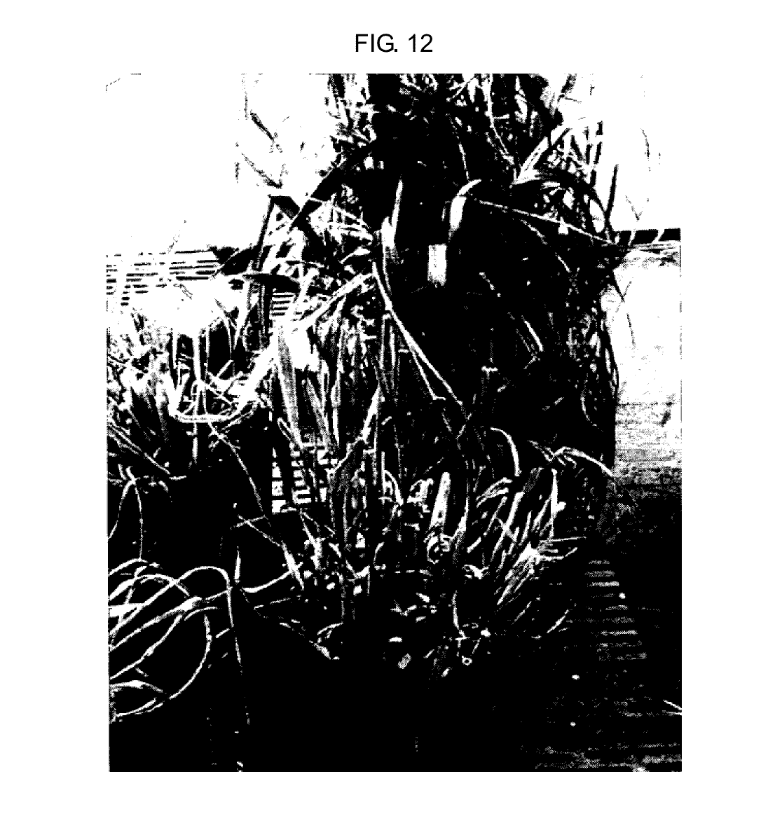

Using adult plants, all the material was eliminated from the soil surface, verifying at 10 days from the cut, the appearance of new leaves on the surface, similar to a sprout. The detail is shown in FIG. 12.

Due to this formation, all the studied specimen show a perennial habit, meeting with all the vegetative and reproductive stages known in Triticum aestivum, but at the end of their cycle they will have a sprout capacity from a reserve zone.

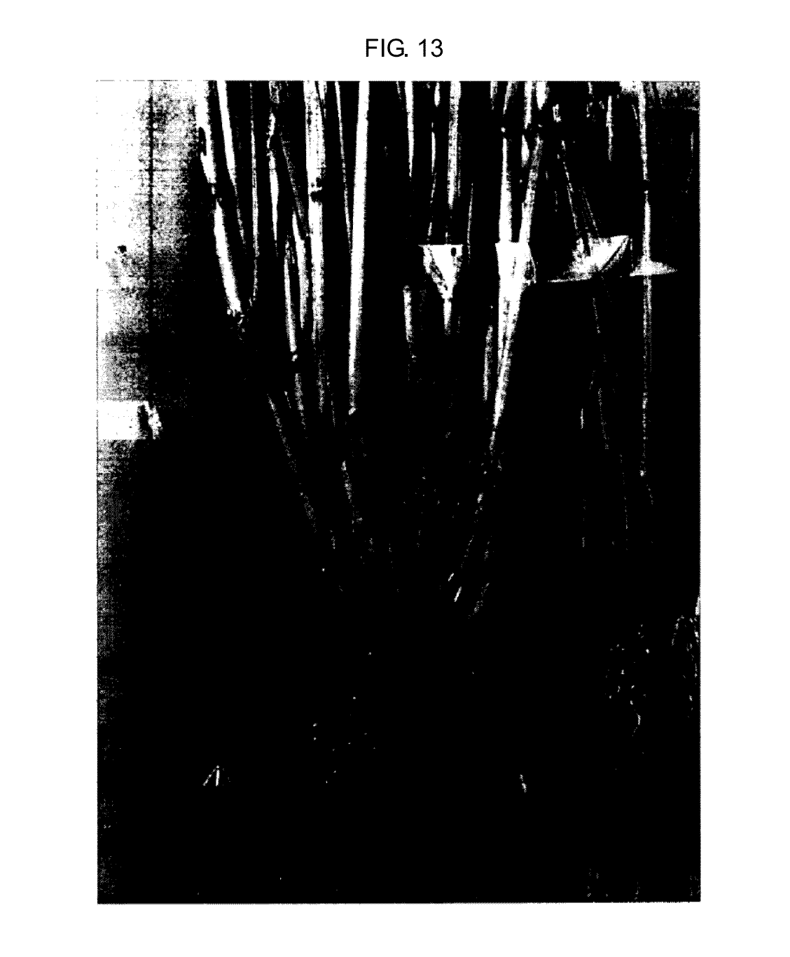

The reeds are plurinodes, with glabrous knots (FIG. 13: pictures of reeds and knots).

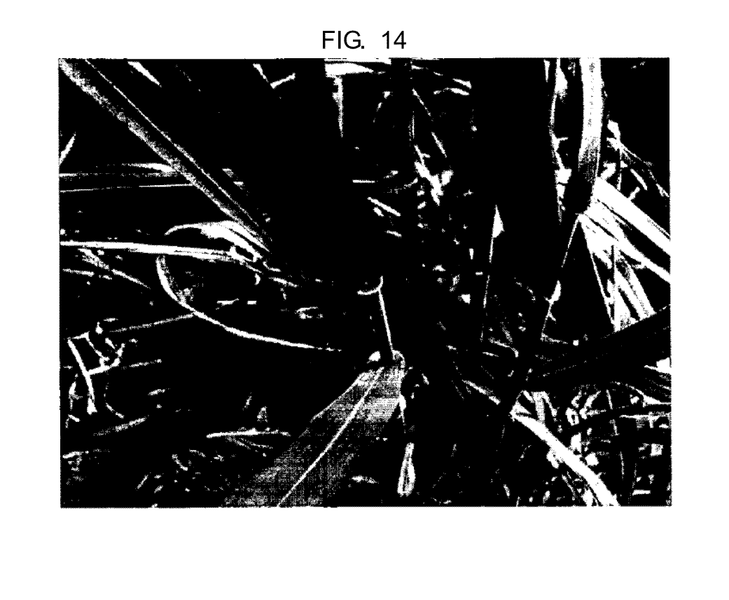

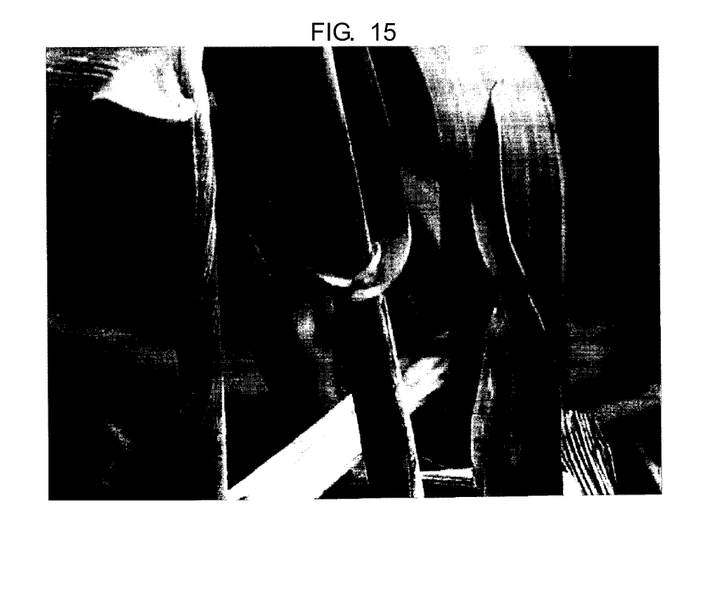

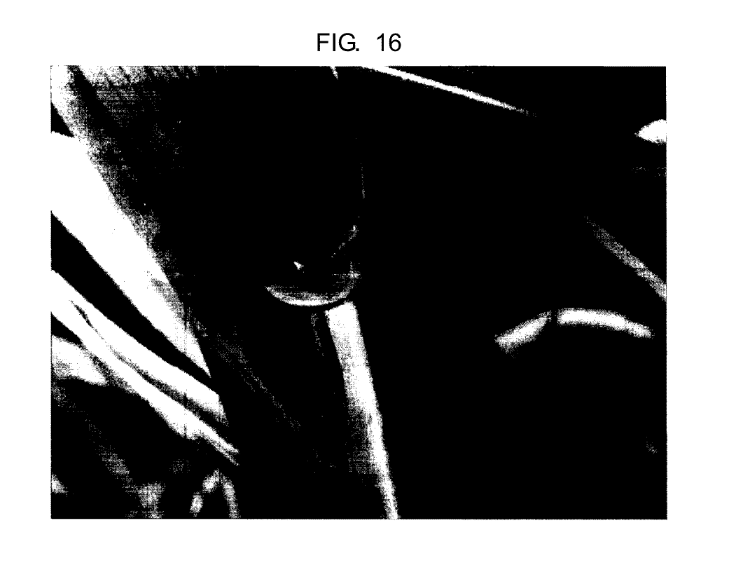

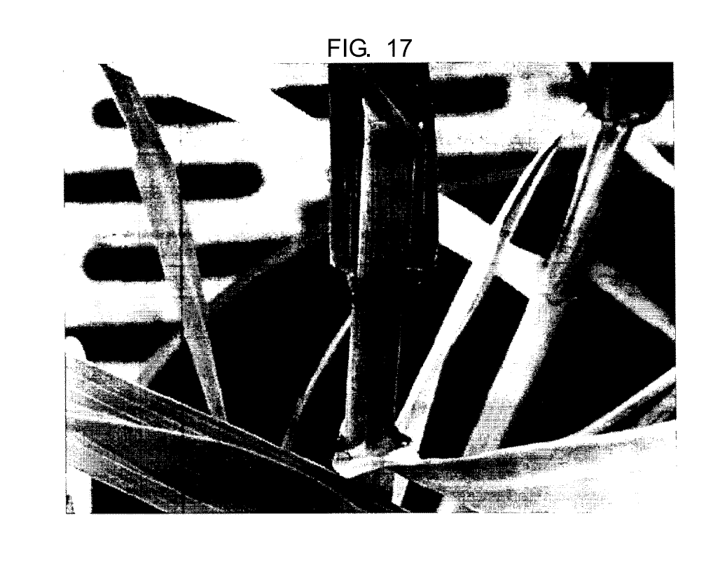

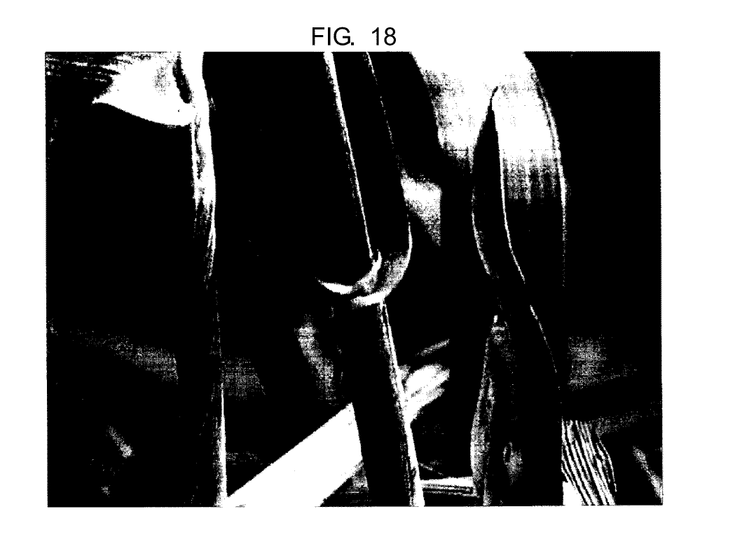

The ligule is membranous from 2-2.5 milimeters (FIG. 14: pictures of ligules). The auricles and laminas are of two types:
Green auricles of 50 milimeters, scantly pilose (FIG. 15: pictures of green auricles), corresponding with flat laminas, glabrous, smooth edges, with a marked central vein and from 16 to 39 centimeters long by 1.5 to 3.0 centimeters wide (FIG. 16: picture of laminas with smooth edge).
Auricles with antocianic pigmentation, of 50 milimeters, glabrous (FIG. 17: pictures of antocianic auricles), corresponding with flat laminas, glabrous, with serrated edge, marked central vein and from 16 to 39 centimeters long by 1.5 to 3.0 centimeters wide (FIG. 18: picture of laminas with serrated edge).

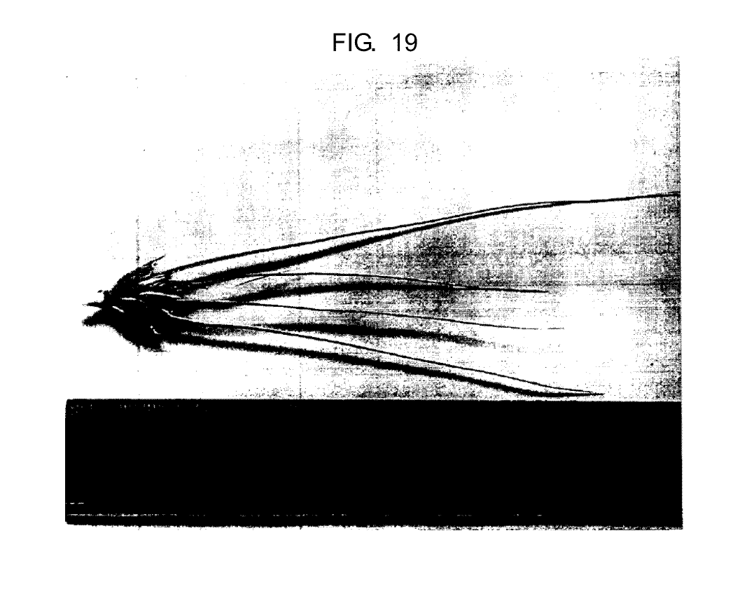

The spikelets have a disposition about the insertion of the flowers with their glumelas at the shaft of the same having a pine form, different from the usual spikelets of Triticum aestivum having an “open hand” form (FIG. 19: picture of spikelets). The number of spikelets in the same spike vary from 16 to 34 spikelts/spike, the basal spikelets having not less that 2 grains, 3 to 4 grains in almost all of them, and in some cases, 6 developed grains.

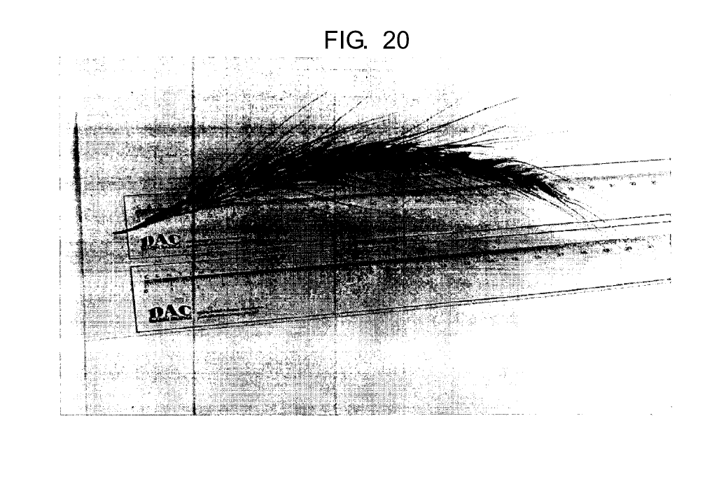

The spikes are straight, having 15-18 centimeters long. Due to the disposition of their spikelets, the spike does not have the small-tall-small size form, as is the case of the traditional spike of Triticum aestivum, but is similar to a spike of cylindrical form from the base to the upper end (FIG. 20: picture of spikes showing the cylindrical structure).

The spike production was not uniform: the first plants spiked on the 30^{th} of October and the last ones the 21^{st} of November. When this period of time is divided in thirds, in the first third 11.5% of the plots spiked, in the second third 55% of the plots spiked and in the rest spiked on the last third.

The color of the pod of the first leaf is light green pubescence absence at the beginning of the formation of the shoots (FIG. 21: picture of pods).

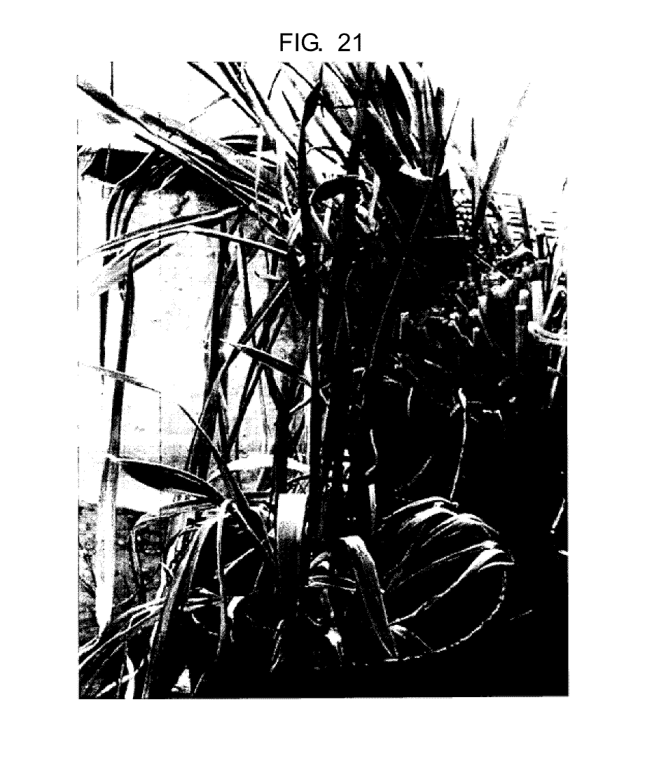

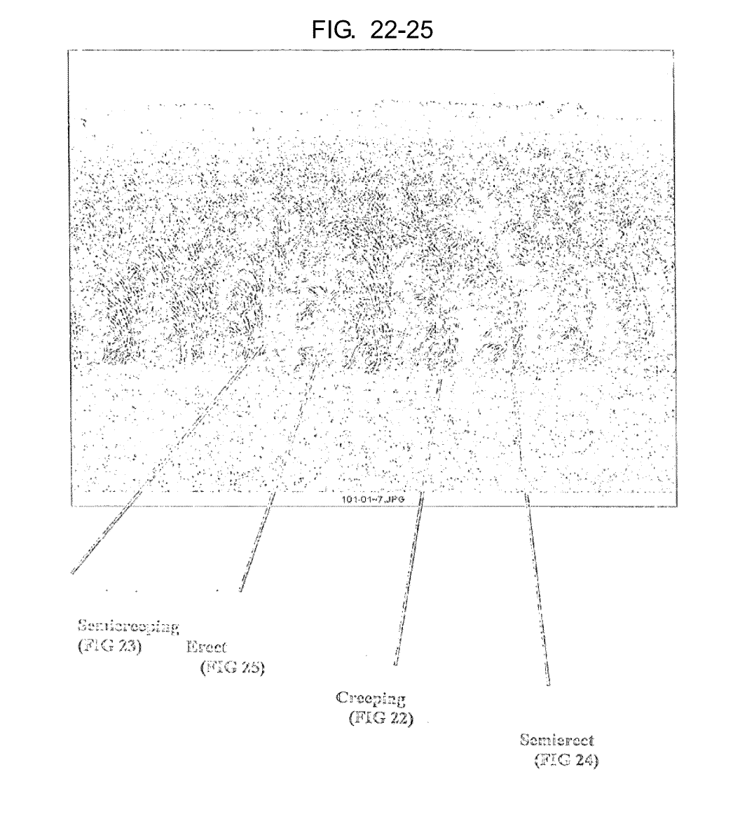

The vegetative appearance is variable presenting all the states: of 1056 plants counted, 21 (2.0%) presented a creeping appearance; 45 (4.3%) presented semi-creeping appearance; 980 (92.8%) presented semi-erect; and 10 (0.9%) presented an erect appearance (FIGS. 22, 23, 24, and 25: pictures of the four appearances).

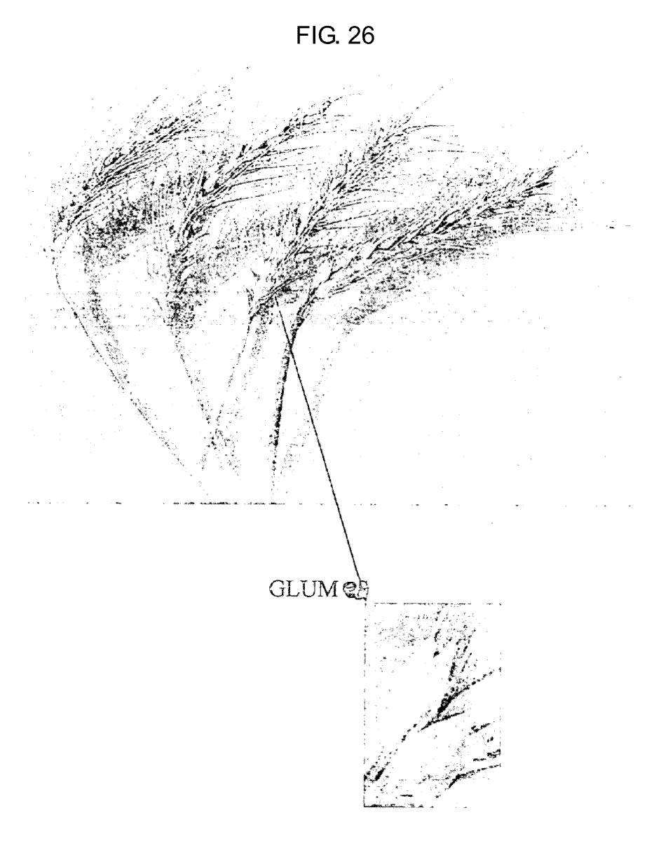

The glumes, main parameter of the possible size of a grain, go from 10 millimeters to 13 millimeters long (FIG. 26: pictures of glumes).

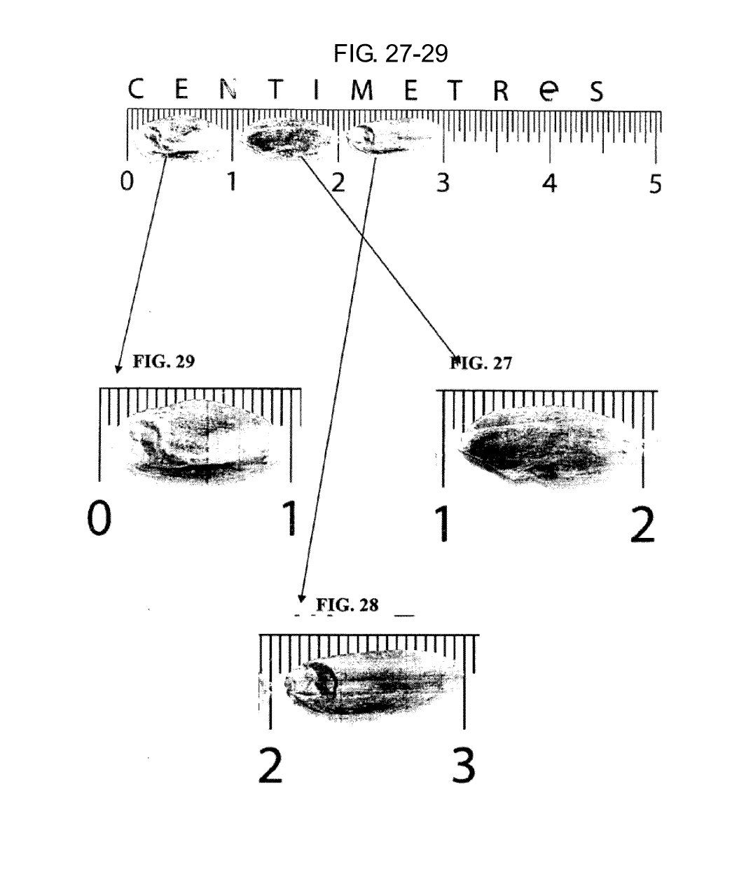

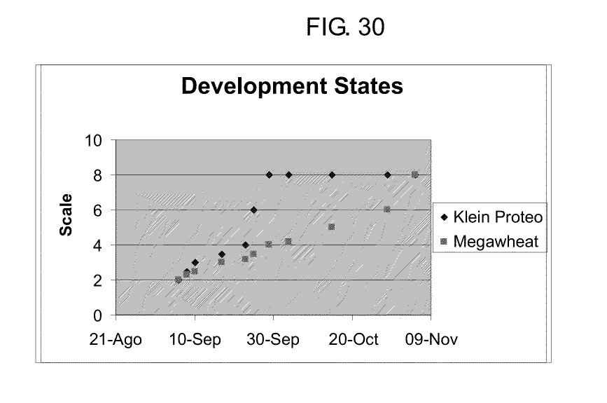

The caryopsis are, in general, red, with isolated cases in amber (FIG. 27: red caryopsis; FIG. 28: amber caryopsis). The size of the caryopsis show great variability, from 7.5×3.0×3.0 millimeters (long×wide×thickness), to 10.0×4.5×4.0 millimeters (FIG. 29: size of caryopsis).

The weight of 1000 grains goes from 55 grams to 80 grams.

The behavior with respect to illnesses produced by fungus or bacteria showed a wide variability, tending in all cases to the higher levels of resistance/tolerance more than to susceptibility.

Comparing the Megawheat plants against a witness variety of Triticum aestivum with commercial name “Klein Proteo”, it can be observed in the following square and figure that both reached at the same time the double ridge, i.e., the visible beginning of the spikelets formation (September 10th , using the Gardner Scale (Gardner et al., 1985). However, “Klein Proteo” reached the terminal spikelet state (state 8 of the Gardner scale), on September 20th, while the Megawheat plants have only formed half of the spikelets approximately (state 4 of the Garder scale). This different rhythm of formation of spikelets can be related to the final number of spikelets/spikes; the slower the process, the higher the final number of spikelets. In this way, “Klein Proteo” had an average of 5 plants, 14 spikelets per spike the material in study, 20.

| | Klein Proteo | Megatrigo |
|---|---|---|
| 06-Sep | 2 | 2 |
| 08-Sep | 2.5 | 2.3 |
| 10-Sep | 3 | 2.5 |
| 17-Sep | 3.5 | 3 |
| 23-Sep | 4 | 3.2 |
| 25-Sep | 6 | 3.5 |
| 29-Sep | 8 | 4 |
| 04-Oct | 8 | 4.2 |
| 15-Oct | 8 | 5 |
| 29-Oct | 8 | 6 |
| 05-Nov | 8 | 8 |

2) Productive Potential of Megawheat

In order to prove the productive potential of Megawheat, a Comparative Yield Test was made by means of the experimental design of Random Complete Blocks, which was treated statistically by means of a Variance Analysis and later by a test of multiple comparison of means by means of the LSD test of Fisher, by means of the software Infostat (Infostat, 2004).

The Variance Analysis allows testing hypothesis related to the position parameters (esperanza) of two or more distributions. The hypothesis that is submitted to test generally is established with respect to the averages of the populations in study or to each one of the treatments evaluated in an experiment:

H0: μ1=μ2= . . . =μa con i=1, . . . , a

where a=number of treatments, which in the case to be analyze is represented by the most important culture samples of the long cycle wheat marketed in Argentina and the unknown treatment, in this case the Megawheat seeds.

By means of the Variance Analysis the total variability in the sample (the addition of the total squares of the observations) is decomposed in components (sum of the squares) each one related to a known variation source (Nelder, 1994; Searle, 1971, 1987).

One of the main objectives in the planning of an experience, following an experimental design, in the error or variability reduction between experimental units that receive the same treatment, with the purpose of increasing precision and sensitivity at the inference moment, for example that related to the comparison of treatment effects (Snedecor, 1956; Snedecor and Cochran, 1967).

The experimental design is a strategy of the combination of the treatment structures (interest factor) with the structure of experimental units (plots, individuals, plant pots, etc.), in a way that the alterations in the responses, at least in some subgroup of the experimental units, may be attributed only to the action of the treatments except because random variations. This way, it is possible to contrast (compare) averages of treatments or linear combinations of averages of treatments with the least “noise” possible.

The experimental design selected to determine the productive potential of Megawheat was the Random Complete Blocks.

The selection of this design parts from the assumption that when there is variability between the experimental units, in this case different wheat cultures of long cycle and the unknown, the groups of homogeneous experimental units may be seen as blocks to implement the experimental strategy known as Design in Blocks. The block principle indicates that the experimental units in each block or group should be similar (homogeneity inside the block) and the blocks should be different (heterogeneity between 0 blocks). The blocking or grouping of experimental material should be such that the experimental units in a block are as homogeneous as possible and the blocks should be design so that the differences between the experimental units are explained, mainly, by the differences between blocks. When the design has been conducted in blocks, the model for each observation should include a term that represents the effect of the block to which the observation belongs (Little and Jackson Hills, 1978; Infostat, 2004).

In this way, it is possible to eliminate from the comparisons between the units that receive the different treatment, variations due to the structure present between plots (blocks).

If each block has as much experimental units as treatments and all the treatments are randomly assigned in each block the design is called Design in Complete Blocks at Random (DBCA by it abbreviation in Spanish). The design is in complete blocks because inside each block there are all the treatments, and at random because inside each block the treatments are assigned to the plots at random. All the plots in the same block have the same probability of receiving any of the treatments. The variation between blocks does not affect the differences between the means since each treatment appear the same number of times in each block. This design allows a greater precision than the completely random, when its use is justified by plot structures.

According to the software used, the following linear model can be postulated to explain the variation of the response, that in the block j receives the treatment i, obtained in a block design with only one treatment factor (Infostat, 2004):

Yij=μ+τi+βj+εij con i=1, . . . , a

Where μ is the general mean, τi is the effect of the i-th treatment, βj is the effect of the j-th block (j=1, . . . , b) and εij is the random error associated to the observation Yij.

Commonly the error terms are assumed normally distributed with esperanza zero and common variance σ2.

Another assumption accompanying the model specification for a block design is the addition (no interaction) of the block and treatments effects.

The verification of the assumptions made about the error term and the comparison of the means of the treatments generally go with this type of solution.

Finally, when the effects of a factor in the ANAVA are considered as valid, a multiple comparison of means test in implemented. From the sample means in each one of the compared distributions, the means of all the treatments are compared. To analyze the differences of “a pares” between the compared distribution means, is possible to make a great variety of tests a posteriori or multiple comparison tests, selecting the LSD test of Fisher due to its tradition in the comparison of means in treatments between cultures (Hsu, 1996; Hsu and Nelson, 1998).

The LSD test, acronym of Least Significant Difference, compares the differences observed between each pair of sample averages with the critical value corresponding to the T test for two independent samples. When working with balanced data, as in the case of the design used, this test is equivalent to the least significant difference test of Fisher, for the whole comparison of means of principal effects. The test does not adjust the level of simultaneous significance, for this reason the rate of error per experiment may be higher at a nominal level, increasing as the number of treatments to evaluate increase.

The Comparative Test of Yield was carried out in La Dulce, party of Necochea, province of Buenos Aires, Republic of Argentina, located at 38° 17′ Lat. S-59° 12′ Long. W.

Fifteen cutivars participated, 10 of which were witness cultures of long cycle of a vast diffusion in Argentina, 4 non-commercial experimental cultures, and the Megawheat seeds.

The culture list of the test as the following:

| Treatment No. | Company | Culture or seeds |
|---|---|---|
| 1 | RELMO | INIA TIJETERA |
| 2 | ACA | 303 |
| 3 | BUCK | ARRIERO |
| 4 | BUCK | FAROL |
| 5 | BUCK | GUAPO |
| 6 | KLEIN | ESCORPION |
| 7 | KLEIN | ESTRELLA |
| 8 | KLEIN | MARTILLO |
| 9 | KLEIN | SAGITARIO |
| 10 | KLEIN | ESCUDO |
| 11 | THOMAS | EXP LUMB 21 |
| 12 | THOMAS | EXP LUMB 22 |
| 13 | THOMAS | EXP LUMB 23 |
| 14 | THOMAS | EXP LUMB 24 |
| 15 | MEGASEED | MEGAWHEAT |

Four trials (blocks) were made with the following random number of treatments:

| | | | | | | | | | | | | | | | |
|---|---|---|---|---|---|---|---|---|---|---|---|---|---|---|---|
| Block I | 1 | 2 | 3 | 4 | 5 | 6 | 7 | 8 | 9 | 10 | 11 | 12 | 13 | 14 | 15 |
| Block II | 8 | 1 | 9 | 2 | 10 | 3 | 11 | 4 | 12 | 5 | 13 | 6 | 14 | 7 | 15 |
| Block III | 11 | 6 | 1 | 12 | 7 | 2 | 13 | 8 | 3 | 14 | 9 | 4 | 15 | 10 | 5 |
| Block IV | 13 | 9 | 5 | 1 | 14 | 10 | 6 | 2 | 15 | 11 | 7 | 3 | 12 | 8 | 4 |

The treatments were sowed in plots of 7 furrows of 5.50 m long and 0.20 m between furrows. The five central furrows were grown to leave a final size of plot of 5.5 m². The final weight of the plot in grams/plot was converted to Kg/Ha.

| | GRAMS | | | | | Kg/Ha | | | | |
|---|---|---|---|---|---|---|---|---|---|---|
| Treatment | B I | B II | B III | B IV | TOTAL | B I | B II | B III | B IV | Kg/Ha |
| RELMO INIA | 2981 | 3235 | 3195 | 3028 | 12439 | 5420 | 5882 | 5809 | 5505 | 5654 |
| TIJETERA | | | | | | | | | | |
| ACA 303 | 4389 | 3902 | 3862 | 3802 | 15955 | 7980 | 7095 | 7022 | 6913 | 7252 |
| BUCK | 3697 | 3321 | 3216 | 3966 | 14200 | 6722 | 6038 | 5847 | 7211 | 6455 |
| ARRIERO | | | | | | | | | | |
| BUCK FAROL | 4529 | 4265 | 4161 | 3986 | 16941 | 8235 | 7755 | 7565 | 7247 | 7700 |
| BUCK | 3837 | 3979 | 3811 | 3886 | 15513 | 6976 | 7235 | 6929 | 7065 | 7051 |
| GUAPO | | | | | | | | | | |
| KLEIN | 3711 | 2882 | 3398 | 3308 | 13299 | 6747 | 5240 | 6178 | 6015 | 6045 |
| ESCORPION | | | | | | | | | | |
| KLEIN | 3963 | 4404 | 3737 | 3954 | 16058 | 7205 | 8007 | 6795 | 7189 | 7299 |
| ESTRELLA | | | | | | | | | | |
| KLEIN | 3547 | 2140 | 2718 | 2774 | 11179 | 6449 | 3891 | 4942 | 5044 | 5081 |
| MARTILLO | | | | | | | | | | |
| KLEIN | 2984 | 3147 | 3231 | 3246 | 12608 | 5425 | 5722 | 5875 | 5902 | 5731 |
| SAG ITARIO | | | | | | | | | | |
| KLEIN | 4852 | 4498 | 4195 | 3939 | 17484 | 8822 | 8178 | 7627 | 7162 | 7947 |
| ESCUDO | | | | | | | | | | |
| EXP LUMB 21 | 4151 | 4569 | 3934 | 4190 | 16844 | 7547 | 8307 | 7153 | 7618 | 7656 |
| EXP LUMB 22 | 3866 | 4171 | 3785 | 3799 | 15621 | 7029 | 7584 | 6882 | 6907 | 7100 |
| EXP LUMB 23 | 4235 | 3668 | 3718 | 4011 | 15632 | 7700 | 6669 | 6760 | 7293 | 7105 |
| EXP LUMB 24 | 1692 | 1663 | 1650 | 1460 | 6465 | 3076 | 3024 | 3000 | 2655 | 2939 |
| MEGAWHEAT | 5040 | 5315 | 5180 | 5300 | 20835 | 12600 | 13288 | 12950 | 13250 | 13022 |

The variance analysis about the design used showed the following results:

Variance Analysis

Variable

| | N | R² | R² Aj | CV |
|---|---|---|---|---|
| Kg/Ha I | 60 | 0.96 | 0.95 | 7.07 |

Variance Analysis Table (SC type III)

| F.V. | SC | gl | CM | F | p-valor |
|---|---|---|---|---|---|
| Model | 253600564.9 | 17 | 14917680.3 | 61.99 | <0.0001 |
| MATERIAL | 252024487.8 | 14 | 18001749.1 | 74.81 | <0.0001 |
| REP | 1576077.14 | 3 | 525359.05 | 2.18 | 0.1042 |
| Error | 10106985.41 | 42 | 240642.51 | | |
| Total | 263707550.3 | 59 | | | |

Test: LSD Fisher
Alfa: = 0.05
DMS: = 700.01910
Error: 240642.5097
gl: 42

| MATERIAL | Means | n | | | | | | | | |
|---|---|---|---|---|---|---|---|---|---|---|
| MEGAWHEAT | 13022.00 | 4 | A | | | | | | | |
| KLEIN ESCUDO | 7947.27 | 4 | | B | | | | | | |
| BUCK FAROL | 7700.45 | 4 | | B | C | | | | | |
| EXP LUMB 21 | 7656.36 | 4 | | B | C | | | | | |
| KLEIN | 7299.09 | 4 | | B | C | | | | | |
| ESTRELLA | | | | | | | | | | |
| ACA 303 | 7252.27 | 4 | | B | C | | | | | |
| EXP LUMB 23 | 7105.45 | 4 | | | C | D | | | | |
| EXP LUMB 22 | 7100.45 | 4 | | | C | D | | | | |
| BUCK GUAPO | 7051.36 | 4 | | | C | D | | | | |
| BUCK | 6454.55 | 4 | | | | D | E | | | |
| ARRIERO | | | | | | | | | | |
| KLEIN | 6045.00 | 4 | | | | | E | F | | |
| ESCORPION | | | | | | | | | | |
| KLEIN | 5730.91 | 4 | | | | | | F | G | |
| SAGITARIO | | | | | | | | | | |
| RELMO INIA | 5654.09 | 4 | | | | | | F | G | |
| TIJETERA | | | | | | | | | | |
| KLEIN | 5081.36 | 4 | | | | | | | G | |
| MARTILLO | | | | | | | | | | |
| EXP LUMB 24 | 2938.64 | 4 | | | | | | | | H |

Different letters indicate significative differences(p <= 0.05)

Conclusion: The carried out test showed a Variability Coefficient of 7.07, totally acceptable in the usual parameters of tests of this characteristics. The yield in Kg/Ha of the unknown treatment Megawheat significantly exceeded all the remaining test treatments. The means contrast using the LSD test for an alpha of 0.05 was 700.01910 Kg/Ha, indicative of the wide superiority in yield expressed in Kg/Ha of the unknown treatment MEGAWHEAT above the remaining test treatments.

### INDUSTRIAL QUALITY

The quality of the wheat flour is one of the essential factors that categorize each variety destined to specific industrial uses. According to the information provided by the Argentina Association PROTRIGO (AAPROTRIGO, 2004), the international demand is getting more demanding: wheat of certain quality and industrial aptitude are necessary for the manufacture of some products in which affect the final quality and most of the consumer acceptance. Quality has become a predominant factor in every commercial transaction.

In the international commerce quality wheat has a differential price for the cost of using an adequate technology and production handling, to obtain getting it to the industry and to export it with the quality which was produced. Countries that do not segregate wheat accept lower market prices that generally are near the value of the fodder wheat. Because the market price is in function of the grain quality, those countries that have wheat classification and segregation systems have comparative advantages with respect to those countries that don't have those systems since, in these cases, the comparative advantage is lost when mixed and is offer to the international market as commodities.

The classification of the wheat production by varieties group and protein is a factor that contributes to improve the profitability of all the participants in the agro-food chain, from the producers to the best demand satisfaction of the industry and exportation.

According to the information of the Secretary of Agriculture, Livestock, Fish and Food of the Republic of Argentina (SAGPyA, (by its Spanish abbreviation), 2004), the general parameters of the industrial quality of wheat flour are: protein percentage, wet or dry gluten percentage, or the ratio between them; enzymatic activity measured as falling) number, ash content and granulometry.

The breading parameters of flour are: water absorption, dough development, dough stability, dough fall, dough force (W), resistance (P), dough extendibility (L) and the ratio (P/L).

A survey made by the SAGPyA between professionals from the National Institute of Industrial Technology of the Republic of Argentina (INTI), private companies and the Professional Pasta Association, the following table shows the industrial quality parameters required by the different industries:

| | | | | | | % | Wet | Gluten | |
|---|---|---|---|---|---|---|---|---|---|
| | % Protein | W | P | L | P/L | Ashes | gluten | Index | Stability |
| Sliced | | 330/370 | 100/110 | 100/130 | 0.8/1 | <1.7% | 28/30% | | |
| French | | | | | | | | | |
| Bread | | | | | | | | | |
| Soft, thin | | >280 | | | 0.9/1.1 | | 30% | >0.6% | 20 minutos |
| crusted | | | | | | | | | |
| bread | | | | | | | | | |
| Pie crusts | | >240 | | | 0.9/1.0 | <0.55% | >26% | | |
| Fresh Pasta | >12% | >270 | | | >0.9 | <0.5% | >33% | | 17 |
| cookies | | 200/400 | 70/80 | 80/100 | 0.8 | <1.8% | 27% | | |
| Sweet | | 250 +/− 20 | 80/100 | 80/100 | 1 | | 20/23% | | |
| cookies | | | | | | | | | |
| Grisines | | 120 | 50 | 100 | 0.5 | | | | |
| Pastas de | >12/13% | 180/350 | 110/120 | 50 | 1.2/2.5 | <0.55/0.75% | 28/34 | | |
| candela | | | | | | | | | |

According to the Argentine Association PROTRIGO (AAPROTRIGO, 2004), the bread quality of wheat and in short the quality of bread, depends on the following factors:
The genetic aptitude of the variety that marks the reachable potential.
The climate conditions during the farming cycle.
The resources of the selected soil for the cultivation.
The technological resources used for the cultivation.
The post-harvest handling of the production in the field, the stock and the terminal elevators.
The industrial process of flour transformation.
The industrial process of bread transformation.

In the case of Australia, the wheat of said country are classify according the following:
Prime hard: white wheat corrector of excellent quality, with a guarantee of a minimum protein level of 13% and 14%.
Hard: white wheat that is segregated to a minimum protein level of 11.5%.
Premium white: is a mixture of selected varieties, with a guarantee of a minimum protein level of 10%.
Noodle: wheat appropriate for the production of white saline noodles, mixture exported to Japanese and south-Korean markets.
Soft Wheat: mixture of soft wheat varieties, segregated to guarantee a maximum protein level of 9.5%.
Durum: selected varieties of amber and vitreous wheat with a minimum protein level of 13%

In the case of Canada, the wheat of said country is classified according to the following:
Canada Western Extra Strong: is a hard red spring wheat with stronger gluten for mixture purpose and special breads.
Canada Western Red Spring: is a hard wheat with a superior quality for bakery. Different minimum levels of protein are guarantee: 12.5%, 13.5% and 14.5%.
Canada Western Red Winter: is a hard wheat that provides low to medium protein levels from and a medium force gluten.
Canada Western Amber Durum: is a hard wheat with high yield of semolina for the production of pasta.
Canada Prairie Spring Red: is a semi-hard wheat with an average protein between 11 and 12%.
Canada Prairie Spring White: is a white wheat with high yields of protein levels between 10.5% and 11.5%.
Canada Western Soft White Spring: is a soft wheat with a low protein content (between 9% and 10%) for the production of cookies.
Canada Western Feed: is a wheat with a high quality for forage, high protein content.

In the case of the United States, the diverse varieties of winter and spring wheat are group in eight official classes. The classes of each variety are determined by its hardness, its grain color and the sow time. Each class of wheat has its own standard characteristics with respect to grinding, baking and other food uses, namely;
Hard Red Winter: is an important baking wheat that represents 40% of the American production and exportation. It has a mildly high content of protein; generally the average is between 11 and 12%.
Hard Red Spring: is a baking wheat with the higher protein content, generally 13 and 14%. Represents 20% of the American exportations. It has three subclasses depending on the darkness, the hardness and clarity of the grain.
Hard White: is the newest kind produced. Is used mainly in the domestic American market for the production of noodles.
Soft White: is used for light breads, cookies and noodles. Is a low protein wheat, generally with a level of 10%. There are three subclasses.
Soft Red Winter: Is the wheat with a higher yield, but with a relatively low protein, generally 10%.
Durum: Is the hardest wheat that provides semolina for the production of pasta. It has an amber color. There are three subclasses.
Unclassified Wheat: all of the varieties not included in the other criteria, any other wheat with a color different from red or white.
Mixed Wheat: any mixture of wheat consisting of less than 90% of one class and more than 10% of another.

In Argentina there is no wheat classification system, but there can be mentioned the different types of wheat that can be sow:
Hard wheat (wheat for bread),
Soft wheat (wheat for cookies),
white wheat (wheat for noodles) and
fodder wheat.

The quality of a variety is determined by the amount and composition of the reserve proteins. In this event, it is possible a variety differentiation by Quality Groups based in its genetic characteristics.

The varieties of Group 1 are genetically correctors of others of an inferior quality. When mix with weak wheat the quality is strengthen producing an excellent volume of bread. The varieties of Group 2 are of a very good baking quality that tolerates long periods of fermentation. The varieties of Group 3 are very yielding by of a deficient baking quality. At the same protein level the varieties of Group 1 will be of a better quality than Group 2 and these ones are better than Group 3.

For the formation of the mentioned groups it was taken into account the following parameters: hectolitrical weight, ashes, % wet gluten, W of the alveogram, pharinographic stability and bread volume.

The following is the classification of hard wheat quality proposed for Argentina by the Argentine Assiciation PROTRIGO and the National Institute of Agricultural Technology (INTA):

Classes of Hard Wheat
TDA 1 Superior (Hard Argentine Wheat One). Formed by varieties of Group 1 of quality with 3 bands of protein:
   TDA1 with protein band between 10.5% to 11.5%
   TDA1 with protein band between 11.6% to 12.5%
   TDA1 with protein of more than 12.5%
TDA 2 Special (Hard Argentine Wheat Two). Formed by varieties of Group 1 and 2 and with 3 protein bands:
   TDA 2 with protein band between 10.0% to 11.0%
   TDA 2 with protein band between 11.1% to 12.0%
   TDA 2 with protein higher than 12.0%
TDA 3 Standard (Hard Argentine Wheat Three). Formed by varieties of Group 3 with 2 protein bands:
   TDA 3 with protein band between 10.0% to 11.0%
   TDA 3 with protein higher than 11.0%

In all the cases the protein bands are use to secure a functionality and do not mean any compensation. The compensations should be accorded in the respective buying-selling contracts. The classification by class means a different use for each one of them (corrector, French bread, soft, thin-crusted bread, cookies, etc.).

With respect to the Megawheat species, the baking quality analysis carried out in Cereal Chair of the Agronomy Faculty of Azul (National Central University), showed with respect to electrophoresis SDS-PAGE, about individual grains, the following proteinic pattern of glutenins of a high molecular weight:
GluA1:2*
GluB1:7+9
GluD1:5+10

This glutenin patterns corresponds to varieties of a very good baking quality.

It was found in the gliadine pattern, the introgression with rye, that gives the material a good behavior with respect to some illnesses.

With respect to the characteristics of industrial quality, the following results were found:
Gluten force: The sedimentation test was made according to the Dick and Quick technique (1983). The test was carried out with 1 g of wheat flour and six trials. The sediment height was 80 mm, which is comparable to commercial bread wheat varieties of good bread quality.
Alveogram: This rheologic test, carried out on white flour of the material in study, obtained the following values:
   W(baking force): 387
   P(tenacity): 107
   L(extensivity): 99
   P/L: 1.08
Weight of one thousand grains: 55-80 g.
Protein Content: 16.8%. (Comparatively, the protein contents of some varieties of bread wheat from Argentina are the following: Prointa Gaucho, 12.0%; Thomas Aconcagua, 10.30%; Thomas 796; 12.80%; Klein Don Enrique, 12.0% and Buck Halcon, 12.50%).

Conclusion: These parameters correspond to a balanced wheat, of a very good baking aptitude, that have the requirements to be considered TDA 1 Superior, and also is placed in the superior scale of quality for the cited quality standards for Australia, Canada and the United States.

4) Mitotic Count

The cytological analysis were carried out in the Wheat Precise Genetic Stocks of the John Innes Centre, Norwich Research Park, Colney, Norwich, United Kingdom.

The analyses revealed that the mitotic count in metaphase of cells in division from the end of the root of Megawheat was 2n=42 chromosomes.

Although all the analyze plants had a constitution of 2n=42 chromosomes, in some cases segregations were observed for one of the satellites normally visible of the wheat genus that may be the 1B or 6B. Some plants showed 4 satellites, others 3 satellites, others 2 satellites and, in some cases, it was not possible to determine the number of satellites.

5) Molecular Characterization

The molecular characterization of cultivation is one of the varied applications of the molecular genetic. The same allows the detection of differences in the DNA of individual plants.

The molecular characterization can be a tool in itself, by means of the identification of specific molecular markers for the cultivation under analysis. The molecular markers can be used to prove the level of genetic diversity between cultivates. Other times, the studies try to identify these markers relative to their link to specific genes of value in the culture.

A series of molecular markers have been developed in order to apply them to molecular characterization studies. The markers RFLP (Restriction Fragment Length Polymorphisms) were the firsts developed and showed to be of great used for the molecular characterization of diverse germplasms including wheat. With the later development of PCR (Polymerase Chain Reaction) technology a new series of markers emerged. The first ones were the RAPD (Random Amplified Polymorphic DNA), which gain popularity by their ease of use and low cost compared to the RFLP. However, this technique showed serious weaknesses related to the lack of reproducibility of the results between different laboratories. The AFLP (Amplified Fragment Length Polymorphisms) markers which combine the amplification PCR of specific fragments of DNA previously digested by endonuclease restriction, showed its high discriminatory capacity between cultivars and reproducibility. Also, the SSR (Simple Sequence Repeats) markers combine the discriminatory ability of the RFLP with the relative easiness of use of the RAPDs (Rapela, 2000; Hoisington et al., 2002).

The SSR markers have gain a rapid acceptance in academic and industrial fields due to their co-dominant nature, reproducibility, high level of polymorphisms detected, high information content, average cost, low technical difficulty, alternative use of radioisotopes, and the same have been used for varietal fingerprinting, genetic diversity studies, qualitative gene tagging, QTL mapping and comparative mapping (Rapela, 2000; Manifesto et al., 2001; Hoisington et al., 2002). The SSR analysis consists in an amplification by PCR using primers of 18 to 25 pairs of base of longitude, which are specific of the regions that flank the presence of 2 to 4 pairs of bases repeated in tandem. The variation in the number of pairs of bases repeated in tandem determines the differences in the length of the amplified fragments (Rapela, 2000; Manifesto et al., 2001).

For this presentation, the molecular analyses of SSR were carried out in the Genome Laboratory of John Innes Centre, Norwich Research Park, Colney, Norwich, UK.

Of the 200 markers SSR available 22 of them were elected in order to cover 3 sets of 7 chromosomes and both arms of each chromosome and 3 markers of unknown position.

The DNA of coleoptiles of wheat seeds was extracted using the Mini Kit of the plant Qiagen's DNeasy. The loci SSR used in this analysis was developed by IPK Gatersleben (gwm and gdm), an international consortium directed by Agrogene S A, Moissy Cramayal, France (wmc), the John Innes Centre (psp), P. Cregan, Q. Song and Assiciates at the USDA-ARS Beltsville Agriculture Research Station (barc).

The amplification reactions were carried out in a thermocycle Perking Elmer Tetrad (Perkin-Elmer, Norwalk, CR) in reaction mixture of 6.25 u1. Each reaction had 3.175 u1 of Qiagen HotStarTaq Mastermix, 0.2 uM pair of atomic trigger and approximately 20 ng of DNA genomic as a tempering. The amplification products were separated in a sequentiator AB13700 and analyzed using the software Genescan and Genotyper (Applied Biosystems, Warrington, UK). The amplified SSR fragments having a different size by at least 2 bp were considered as different alleles.

In the following Table there are the results of the analyses carried out on samples of two parental cultivars (Thomas 796 and Thomas Aconcagua) and the Megawheat, indicating the size of the amplified allele in pairs of bases, the marker designation SSR, its location (where the number identifies the chromosome, the first letter indicated if it belongs to the A, B, or D genome and the letters L, S, or C indicate if is in the long arm, short or in the central region).

| allele | allele | allele | | | | |
|---|---|---|---|---|---|---|
| size | size | size | | | | |
| (bp) | (bp) | (bp) | marker | location | line no. | line |
| 169.00 | | | barc168 | ? | 1 | Thomas 796 |
| 168.90 | | | barc168 | ? | 2 | Thomas |
| 169.03 | | | barc168 | ? | 3 | Aconcagua |
| | | | | | | Megawheat |
| 128.59 | | | gwm213 | ? | 1 | Thomas 796 |
| 128.73 | | | gwm213 | ? | 2 | Thomas |
| 128.58 | | | gwm213 | ? | 3 | Aconcagua |
| | | | | | | Megawheat |
| 141.63 | | | gwm388 | ? | 1 | Thomas 796 |
| 141.68 | | | gwm388 | ? | 2 | Thomas |
| 141.72 | | | gwm388 | ? | 3 | Aconcagua |
| | | | | | | Megawheat |
| 274.16 | | | barc083 | 1AL | 1 | Thomas 796 |
| 274.10 | | | barc083 | 1AL | 2 | Thomas |
| 274.17 | | | barc083 | 1AL | 3 | Aconcagua |
| | | | | | | Megawheat |
| 159.88 | 173.96 | 181.85 | psp3100 | 1BL | 1 | Thomas 796 |
| 159.82 | | | psp3100 | 1BL | 2 | Thomas |
| 181.67 | | | psp3100 | 1BL | 3 | Aconcagua |
| | | | | | | Megawheat |
| 207.00 | 212.76 | | gdm111 | 1DL | 1 | Thomas 796 |
| 206.97 | | | gdm111 | 1DL | 2 | Thomas |
| 207.05 | | | gdm111 | 1DL | 3 | Aconcagua |
| | | | | | | Megawheat |
| 126.24 | | | gwm095 | 2AC | 1 | Thomas 796 |
| 124.28 | | | gwm095 | 2AC | 2 | Thomas |
| 112.45 | | | gwm095 | 2AC | 3 | Aconcagua |
| | | | | | | Megawheat |
| 169.83 | 177.47 | | gwm388 | 2BL | 1 | Thomas 796 |
| 173.60 | | | gwm388 | 2BL | 2 | Thomas |
| 173.75 | | | gwm388 | 2BL | 3 | Aconcagua |
| | | | | | | Megawheat |
| 184.03 | | | barc168 | 2DS | 1 | Thomas 796 |
| 183.92 | | | barc168 | 2DS | 2 | Thomas |
| 181.09 | | | barc168 | 2DS | 3 | Aconcagua |
| | | | | | | Megawheat |
| 119.04 | 133.25 | | wmc264 | 3AL | 1 | Thomas 796 |
| 139.46 | | | wmc264 | 3AL | 2 | Thomas |
| 137.38 | | | wmc264 | 3AL | 3 | Aconcagua |
| | | | | | | Megawheat |
| 184.30 | | | barc164 | 3BL | 1 | Thomas 796 |
| 184.28 | | | barc164 | 3BL | 2 | Thomas |
| 184.25 | | | barc164 | 3BL | 3 | Aconcagua |
| | | | | | | Megawheat |
| 140.25 | | | gdm072 | 3DS | 1 | Thomas 796 |
| 140.05 | | | gdm072 | 3DS | 2 | Thomas |
| 117.59 | | | gdm072 | 3DS | 3 | Aconcagua |
| | | | | | | Megawheat |
| 199.52 | 219.58 | | barc184 | 4AL | 1 | Thomas 796 |
| 199.65 | | | barc184 | 4AL | 2 | Thomas |
| 199.57 | | | barc184 | 4AL | 3 | Aconcagua |
| | | | | | | Megawheat |
| 171.16 | 170.83 | | barc163 | 4BL | 1 | Thomas 796 |
| 165.02 | | | barc163 | 4BL | 2 | Thomas |
| 164.84 | | | barc163 | 4BL | 3 | Aconcagua |
| | | | | | | Megawheat |
| 167.97 | | | wmc457 | 4D | 1 | Thomas 796 |
| 167.93 | | | wmc457 | 4D | 2 | Thomas |
| 168.18 | | | wmc457 | 4D | 3 | Aconcagua |
| | | | | | | Megawheat |
| 273.65 | | | barc141 | 5AS | 1 | Thomas 796 |
| 275.79 | | | barc141 | 5AS | 2 | Thomas |
| 273.62 | | | barc141 | 5AS | 3 | Aconcagua |
| | | | | | | Megawheat |
| 175.40 | | | gwm213 | 5BC | 1 | Thomas 796 |
| 175.35 | | | gwm213 | 5BC | 2 | Thomas |
| 175.40 | | | gwm213 | 5BC | 3 | Aconcagua |
| | | | | | | Megawheat |
| 186.20 | | | barc110 | 5DL | 1 | Thomas 796 |
| 201.02 | | | barc110 | 5DL | 2 | Thomas |
| 186.10 | | | barc110 | 5DL | 3 | Aconcagua |
| | | | | | | Megawheat |
| 246.23 | | | dupw167 | 6AL | 1 | Thomas 796 |
| 246.34 | | | dupw167 | 6AL | 2 | Thomas |
| 246.23 | | | dupw167 | 6AL | 3 | Aconcagua |
| | | | | | | Megawheat |
| 195.80 | 203.58 | | barc134 | 6BL | 1 | Thomas 796 |
| 197.62 | 197.74 | | barc134 | 6BL | 2 | Thomas |
| 191.90 | | | barc134 | 6BL | 3 | Aconcagua |
| | | | | | | Megawheat |
| 166.22 | | | gwm469 | 6DS | 1 | Thomas 796 |
| 166.13 | | | gwm469 | 6DS | 2 | Thomas |
| 166.21 | | | gwm469 | 6DS | 3 | Aconcagua |
| | | | | | | Megawheat |
| 126.19 | | | gwm130 | 7AS | 1 | Thomas 796 |
| 126.12 | | | gwm130 | 7AS | 2 | Thomas |
| 126.21 | | | gwm130 | 7AS | 3 | Aconcagua |
| | | | | | | Megawheat |
| 185.25 | | | barc072 | 7BS | 1 | Thomas 796 |
| 185.19 | | | barc072 | 7BS | 2 | Thomas |
| 185.08 | | | barc072 | 7BS | 3 | Aconcagua |
| | | | | | | Megawheat |
| NULL | | | gwm130 | 7DS | 1 | Thomas 796 |
| NULL | | | gwm130 | 7DS | 2 | Thomas |
| 114.06 | | | gwm130 | 7DS | 3 | Aconcagua |
| | | | | | | Megawheat |
| 220.78 | 220.83 | | barc076 | 7DL | 1 | Thomas 796 |
| 208.84 | | | barc076 | 7DL | 2 | Thomas |
| 221.00 | | | barc076 | 7DL | 3 | Aconcagua |
| | | | | | | Megawheat |

The analysis indicates the presence in the Megawheat of 6 particular SSR alleles that are not found in any of both parents and could indicate the action at a DNA level caused by the used technique of genetic variability.

The SSR alleles for which Megawheat is different from both parents were: psp3100 (1BL), gwm095 (2AC), wmo264 (3AL), gdm072 (3DS), barc134 (6BL) and gwm130 (7DS).

### REFERENCES

AAPROTRIGO, 2004. Calidad Panadera: [symbol] ,de qué depende la calidad del pan? WEB site: http://www.aaprotrigo.org/calidad.htm

Allan, 1987. ‘Wheat“, Chapter 18, Principles of Crop Development, vol. 2, Fehr editor.

Allard, 1960. Principles of Plant Breeding.

American Optometric Association, 2004—www.aoanet.org. Acceso al sitio WEB, octubre 2004.

Curtis, B. C. 2002. In: B. C. Curtis, S. Rajaram and H. Gómez Macpherson (eds.), 2002. Bread wheat: improvement and production. FAO Plant Production and Protection Series.

Dewey, D. R. 1984. The genomic system of classification as a guide to intergeneric hybridisation with the perennial Triticeae. In J. P. Gustafson, ed. Gene manipulation in plant improvement, p. 209-279. New York, N.Y., USA, Plenum Press

Dvorak, J. and H. B. Zhang. 1990. variation in repeated nucleotide sequences sheds light on the phylogeny of the wheat B and G genomes. Proc. Natl. Acad. Sci. USA 87: 9640-9644.

Environmental Protection Agency, 2004—www.epa.gov/ozone Acceso al sitio WEB, octubre 2004.

Gardner, J. S., Hess, W. M. & Trione, E. J. 1985. Development of the young wheat spike: a SEM study of Chinese spring wheat. Am. J. Bot., 72: 548-559.

Heyne (editor), 1987. Wheat and Wheat Improvement.

Hoisington, D., N. Bohorova, S. Fennell, M. Khairallah, A. Pellegrineschi, J. M. Ribaut, 2002. The application of biotechnology to wheat improvement. In: B. C. Curtis, S. Rajaram and H. Gómez Macpherson (eds.), 2002. Bread wheat: improvement and production. FAO Plant Production and Protection Series.

Hsu, J. C., 1996. Multiple Comparisons: Theory and Methods. London: Chapman & Hall.

Hsu, J. C.; Nelson, B., 1998. Multiple Comparisons in the General Linear Model. Journal of Computational and Graphical Statistics, 7(1): 23-41.

InfoStat, Software Estadistico, Manual del Usuario, Versión 2004

Jaaska, V. 1978. NADP-dependent aromatic alcohol dehydrogenases in polyploidy wheats and their diploi relatives—origin and phylogeny of polyploi wheats. Theor. Appl. Genet. 53: 209-217.

Kielbassa, C., L Roza and B. Epe. 1997. Wavelength dependence of oxidative DNA damage induced by UV and visible light. Carcinogenesis, 18, 811-816.

Kihara, H. 1944. Discovery of the DD-analyser, one of the ancestrors of vulgare wheats. Agric. Hort. 19: 13-14.

Kimber, G. and M. Feldman. 1987. Wild wheat: An introduction. P. 99-103. Special Report 353, College of Agric., Univ. of Missouri, Columbia.

Kubitschek, H. E. 1967. Mutagenesis by near-visible light. Science, 155(769):1545-6.

Manifesto, M. M., A. R. Schlatter, H. E. Hopp, E. Y. Suarez, and J. Dubcovsky. 2001. Quantitative Evaluation of Genetic Diversity in Wheat Germplasm Using Molecular Markers, Crop Sci. 41:682-690.

McFadden, E. S. and E. R. Sears. 1946. The origin of Triticum spelta and its free-threshing exaploid relatives. J. Hered. 37: 81-89.

McGinty, L. D. and Fowler, R. G. 1982. Visible light mutagenesis in Escherichia coli. Mutat. Res. 95(2-3):171-81.

Montelone, B. A., 1998. Mutation, mutagens, and DNA repair. Division of Biology, Kansas State University; originally written as a supplement to BIOL400, Human Genetics

Mori, N., T. Terachi, and T. Tsunewaki. 1988. organellar genome differentiation in wild tatraploid wheats, Triticum dicoccoides and T. araraticum. p. 109-114. In: T. E. Miller and R. D. M. Koebner (ed.) Proc. 7th Int. Wheat genet. Symp., Cambridge, England, U.K.

Morrison, L. A.; 0. Riera-Lizarazu, L. Cremieux, and C. A. Mallory-Smith. 2002. Jointed Goatgrass (Aegilops cylindrica Host)×Wheat (Triticum aestivum L.) Hybrids: Hybridization Dynamics in Oregon Wheat Fields, Crop Sci. 2002 42: 1863-1872.

Mujeeb-Kazi, A. and S. Rajaram. 2002. Transferring alien genes from related species and genera for wheat improvement. In: B. C. Curtis, S. Rajaram and H. Górnez Macpherson (eds.), 2002. Bread wheat: improvement and production. FAO Plant Production and Protection Series.

Nelder, J. A., 1994. The Statistics of Linear Models: Back to Basics. Statistics and Computing, 4: 243-256.

Little, T. M.; Jackson Hill, F. 1978. Agricultural Experimentation. New York, John Wiley & Sons, Inc., New York.

Rapela, M. A. 2000. Derechos de propiedad intelectual en vegetales superiores. Editorial Ciudad Argentina, 466 páginas.

Reynolds, M. P., 2002. Physiological approaches to wheat breeding. In: B. C. Curtis, S. Rajaram and H. Gómez Macpherson (eds.), 2002. Bread wheat: improvement and production. FAO Plant Production and Protection Series.

SAGPyA, 2004. Análisis de la cadena de haring de trigo. Secretaria de Agricultura, Ganaderia, Pesca y Alimentos (SAGPyA), Repüblica Argentina, WEB site: http://www.sagpya.mecon.gov.ar/0-3/farinathar trigo/har 02.htm

Searle, S. R. 1971. Linear Models. New York, John Wiley & Sons, Inc., New York

Searle, S. R. 1987. Linear Models for Unbalanced Data. New York, John Wiley & Sons, Inc., New York.

Sears, E. R. 1953. Nullisomic Analysis in Common Wheat. Am. Nat. 87:245-252.

Sears, E. R. 1954. The aneuploids of common wheat. Mo. Agric. Exp. Sta. Res. Bull., 572: 1-58.

Sharp, P. 2004. Targeted mutagenesis in Wheat. In: Quality Wheat: Newsletter of the CRC for Value Added Wheat. May 17, 2004.

Simmonds, 1979. Principles of Crop Improvement.

Sinha, R. P., P. Richter, J. Faddoul, M. Braun and D. P. Häder. 2002. Effects of UV and visible light on cyanobacteria at the cellular level. Photochemical & Photobiological Sciences, 1(8), 553-559.

Snedecor, G. W. 1956. Métodos Estadisticos Aplicados a la Investigación Agricola y Biológica. Compariia Editorial Continental, S.A., Mexico.

Snedecor, G. W.; Cochran, W. G. 1967. Statistical Methods. Ames, Iowa: Iowa State University Press.

Voskanyan, K. Sh. 1999. UV AND VISIBLE LIGHT—INDUCED MUTATIONS IN ESCHERICHIA COLI. Second Internet Conference on Photochemistry anc Photobiology, Jul. 16-Sep. 7, 1999. Internet Photochemistry and Photobiology, Ar International Forum for Virtual Conferences Voskanyan K.Sh. 1990. 633 nm light induces mutations. Studia Biophysica, v.139, 1 (1990) 43-46.

Waines, J. G. and D. Barnhart. 1992. Biosystematic research in Aegilops anc Triticum. Hereditas, 116: 207-212.

Wang. G. Z., N. T. [symbol] , and K. [symbol] . 1997. Plasmon analysis of Triticum (wheat) and Aegilops: PCR-single-stranded conformational polymorphism (PCR [symbol] SSCP) analysis of organellar DNAs. Proc. Natl. Acad. Sci., USA 94: 14570-14577.

Wheat Genetics Resource Center, 2004. Aegilops classification systems: Classification of Aegilops and Amblyopyrum according to the various proposed systems. WEB site: http://www.ksu.edu/wgrc/Taxonomy/taxaeg.html

Xiang Yang, A. 1990. Study of the mutagenic effect of such physical factors as laser on E. coli and of the auxotrophic analysis. Proc. Inc. Conf. On Lasers in the Life Sciences, Jun. 20-23, 1990 China.

1. A method to obtain a wheat plant with improved yield properties wherein said method consists in the generation of genetic variability and said method comprises the following stages:
a. the construction of a wheat plant F1 by means of the crossing of two parents genetically distant and of opposed industrial qualities;
b. the permanent application throughout the whole development of the inflorescence of said plant of a high concentration of sunlight with no spectrum filter by means of 6 mirrored surfaces each one of 1.00 meter long by 0.50 meter high; said mirrored surfaces are mounted over supports in a way that the mirrors point out by their center towards a stick positioned in an equidistant form in the middle of the plot with the plants;
c. the germination of the resulting seeds and the analysis of the descendants for the search of stable variants of a different chromosomal number.

2. A method to obtain a wheat plant according to claim 1, wherein a stable wheat variant is obtained, said variant presents crown root, high production capacity of fertile shoots, long and wide leaves and in some cases finely serrated, leaves having a central vein, shoot capacity, perennial habit, high level of grain productivity, high protein level and industrial qualities similar to the hard wheat of best quality, that qualify to be designated as a new type of wheat commonly called Megawheat.

3. A method to obtain a wheat plant according to claim 1, wherein the productivity level exceeds by 60% the production of conventional wheat.

4. A method to obtain a wheat plant according to claim 1, wherein seeds with a weight over 55 g, preferably over 70 g are obtained.

5. A method to obtain a wheat plant according to claim 1, wherein the step of the analysis of the descendants consists in the assisted selection of genetic markers and comprises the evaluation of the DNA of the plants; mapping with one or more of the following SSR genetic markers psp3100; gwm095; wmo264; gdm072; barc134; gwml 30 and wherein said markers are placed in the positions according to the following detail:

| | location | | | Allele size |
|---|---|---|---|---|
| marker | chromosome | genome | arm | (bp) |
| psp3100 | 1 | B | length | 181.67 |
| gwm095 | 2 | A | central | 112.45 |
| wmc264 | 3 | A | length | 137.38 |
| gdm072 | 3 | D | short | 117.59 |
| barc134 | 6 | B | length | 191.90 |
| gwm130 | 7 | D | short | 114.06 |

6. A method to obtain a wheat plant according to claim 5 wherein a wheat plant is obtained designated Megawheat.

7. A plant or part of a wheat plant, wherein said plant or part of a wheat plant is obtained by a method which consists in the generation of genetic variability and said method comprising the following stages:
a. the construction of a wheat plant F1 by means of the crossing of two parents genetically distant and of opposed industrial qualities;
b. the permanent application throughout the whole development of the inflorescence of said plant of a high concentration of sunlight with no spectrum filter by means of 6 mirrored surfaces each one of 1.00 meter long by 0.50 meter high; said mirrored surfaces are mounted over supports in a way that the mirrors point out by their center towards a stick positioned in an equidistant form in the middle of the plot with the plants;
c. the germination of the resulting seeds and the analysis of the descendants for the search of stable variants of a different chromosomal number.
wherein in the genetic analysis by means of assisted selection of genetic markers, the following SSR genetic markers psp3100; gwm095; wmo264; gdm072; barc134; gwm130 are located in the positions detailed in the following table:

| | location | | | Allele size |
|---|---|---|---|---|
| marker | chromosome | genome | arm | (bp) |
| psp3100 | 1 | B | length | 181.67 |
| gwm095 | 2 | A | central | 112.45 |
| wmc264 | 3 | A | length | 137.38 |
| gdm072 | 3 | D | short | 117.59 |
| barc134 | 6 | B | length | 191.90 |
| gwm130 | 7 | D | short | 114.06 |

wherein said plant belongs to the selected species between Triticum aestivum, T. turgidum, T. timopheevii, T. monococcum, T. zhukovskyi and T. urartu and hybrids of the same, preferably Triticum aestivum; and
wherein said plant or part of a wheat plant has a crown root, high production capacity of fertile shoots, long and wide leaves and in some cases finely serrated, leaves having a central vein, shoot capacity, perennial habit, high level of grain productivity, high protein level and industrial qualities similar to the hard wheat of best quality, that qualify to be designated as a new type of wheat commonly called Megawheat.

8. A method to obtain a wheat plant according to claim 1, wherein in the genetic analysis by means of assisted selection of genetic markers, the following SSR genetic markers psp3100; gwm095; wmo264; gdm072; barc134; gwm130 are located in the positions detailed in the following table:

| | location | | | Allele size |
|---|---|---|---|---|
| marker | chromosome | genome | arm | (bp) |
| psp3100 | 1 | B | length | 181.67 |
| gwm095 | 2 | A | central | 112.45 |
| wmc264 | 3 | A | length | 137.38 |
| gdm072 | 3 | D | short | 117.59 |
| barc134 | 6 | B | length | 191.90 |
| gwm130 | 7 | D | short | 114.06 |

9. A method to obtain a wheat plant according to claim 1, wherein said plant belongs to the selected species between Triticum aestivum, T. turgidum, T. timopheevii, T. monococcum, T. zhukovskyi and T. urartu and hybrids of the same, preferably Triticum aestivum.

10. A plant or part of a wheat plant according to claim 7, wherein said part of a wheat plant is a seed or part of a seed, a pollen, or a plant ovule all of them obtained from a seed.

11. A plant or part of a wheat plant according to claim 7, wherein said part of a wheat plant is a tissue culture of regenerated cells.

12. A plant or part of a wheat plant according to claim 14, wherein a protoplast culture of regenerated cells is obtained from said tissue culture.

13. A plant or part of a wheat plant according to claim 7, wherein said plant is a wheat plant F1 obtained by the crossing of said plants with any plant of the tribe Triticeae.

14. A plant or part of a wheat plant according to claim 16 wherein said plant is the descendant of said plants FI.

15. A plant or part of a wheat plant according to claim 7, wherein said plant is a hybrid plant.

16. A plant or part of a wheat plant according to claim 7, wherein said plant is a plant resistant to anyone of the group composed by pesticides, insects and diseases.

17. A plant or part of a wheat plant according to claim 7, wherein said plant is a plant with a reduced phytate content.

18. A plant or part of a wheat plant according to claim 7, wherein said plant is a plant with a modified fatty acid metabolism.

19. A plant or part of a wheat plant according to claim 7, wherein said plant is a plant with waxy starch or starch with an amylase increase.

20. A method to generate genetic variability in wheat, preferably Triticum aestivum, wherein said method comprises the steps of:
a. the construction of a wheat plant F1 by means of the crossing of two parents genetically distant and of opposed industrial qualities;
b. the permanent application through the whole development of the inflorescence of said plant of a high concentration of sunlight without spectrum filtration;
c. the germination of the resulting seeds and the analysis of the descendants for the search of stable variants of different chromosomal number.

21. A method to obtain a wheat plant according to claim 2, wherein the productivity level exceeds by 60% the production of conventional wheat.

22. A method to obtain a wheat plant according to claim 2, wherein seeds with a weight over 55 g, preferably over 70 g are obtained.

23. A method to obtain a wheat plant according to claim 3, wherein seeds with a weight over 55 g, preferably over 70 g are obtained.

24. A method to obtain a wheat plant according to claim 2, wherein in the genetic analysis by means of assisted selection of genetic markers, the following SSR genetic markers psp3100; gwm095; wmo264; gdm072; barc134; gwm130 are located in the positions detailed in the following table:

| | location | | | Allele size |
|---|---|---|---|---|
| marker | chromosome | genome | arm | (bp) |
| psp3100 | 1 | B | length | 181.67 |
| gwm095 | 2 | A | central | 112.45 |
| wmc264 | 3 | A | length | 137.38 |
| gdm072 | 3 | D | short | 117.59 |
| barc134 | 6 | B | length | 191.90 |
| gwm130 | 7 | D | short | 114.06 |

25. A method to obtain a wheat plant according to claim 2, wherein said plant belongs to the selected species between Triticum aestivum, T. turgidum, T. timopheevii, T. monococcum, T. zhukovskyi and T. urartu and hybrids of the same, preferably Triticum aestivum.

26. A method to obtain a wheat plant according to claim 3, wherein said plant belongs to the selected species between Triticum aestivum, T. turgidum, T. timopheevii, T. monococcum, T. zhukovskyi and T. urartu and hybrids of the same, preferably Triticum aestivum.

27. A method to obtain a wheat plant according to claim 4, wherein said plant belongs to the selected species between Triticum aestivum, T. turgidum, T. timopheevii, T. monococcum, T. zhukovskyi and T. urartu and hybrids of the same, preferably Triticum aestivum.

28. A method to obtain a wheat plant according to claim 5, wherein said plant belongs to .the selected species between Triticum aestivum, T. turgidum, T. timopheevii, T. monococcum, T. zhukovskyi and T. urartu and hybrids of the same, preferably Triticum aestivum.

29. A method to obtain a wheat plant according to claim 6, wherein said plant belongs to the selected species between Triticum aestivum, T. turgidum, T. timopheevii, T. monococcum, T. zhukovskyi and T. urartu and hybrids of the same, preferably Triticum aestivum
